(19) United States
(12) Patent Application Publication
(10) Pub. No.: US 2002/0169296 A1
(43) Pub. Date: Nov. 14, 2002

(54) Linked, sequence-specific DNA-binding molecules

(76) Inventors: Ulrich Laemmli, Onex (CH); Samuel Janssen, Owings Mills, MD (US)

Correspondence Address: Gary J. Gershik, Cooper & Dunham LLP, 1185 Avenue of the Americas, New York, NY, 10036, US

(21) Appl. No.: 09/903,359

(22) Filed: Jul. 11, 2001

Related U.S. Application Data
(63) Continuation-in-part of application No. 09/614,036, filed on Jul. 11, 2000.

(51) Int. Cl. A61K 48/00; C07H 21/04

(52) U.S. Cl. 536/23.1; 514/44

(57) ABSTRACT

The present invention concerns a DNA-binding molecule, capable of sequence-specific binding to the minor groove of double-stranded DNA, characterized in that it comprises at least two sequence specific DNA-binding elements, covalently linked to each other in tandem orientation by an amphipathic, flexible linker molecule, at least one of said DNA binding elements being non-proteinaceous.

The present invention relates to tandemly linked, sequence-specific DNA-binding molecules with high affinity, specificity and binding-site size. The invention also relates to the in vivo, in vitro and ex vivo use of the tandemly linked binding molecules for binding DNA in a sequence-specific manner, and for regulating chromosome and gene function. The invention also concerns the sequence-specific marking of DNA and chromosomes using marked, tandemly linked binding molecules.

Small synthetic molecules that can target predetermined DNA sequences with high affinity and specificity could represent a major breakthrough in molecular biology. Binding of these molecules could serve to locally interact with proteins as well as to deliver a conjugated chemical group such as a fluorescent label, a toxin, or a peptide.

Recently, considerable progress has been made in the synthesis of small molecules composed of heterocyclic organic molecules for example aromatic amino acids such as N-methylpyrrole (Py) and N-methylimidazole (Im). These molecules can bind specific DNA sequences with remarkable affinities (Geierstanger et al., 1994). The pseudo-peptides (polyamides), based on the structure of the naturally occurring antibiotic distamycin, bind DNA in the minor groove as antiparallel dimers (Pelton and Wemmer, 1989).

The sequence-specificity of these compounds depends on the side-by-side pairing of this dimer, where for example, an Im opposite a Py (Im/Py) targets a G-C base pair, a Py/Im recognizes a C-G base pair and a Py/Py pair (or Py alone) is degenerate for both A.T or T.A base pairs (White et al., 1997). Compounds composed of N-methylpyrrole, N-methylimidazole, N-methyl-3-hydroxypyrrole and certain aliphatic amino acids can therefore be designed in such a way that the position of these units in the mostly linear compound determines the sequence of base pairs to which the compound will bind in the minor groove.

Specificity (and affinity) of targeting increases as the binding site size of the compound increases. Currently however, it is difficult to produce compounds that target a sequence that is longer than 5-7 base pairs since with increasing size, the mismatch between these compounds and the DNA also increases.

For example, each pyrrole carboxamide contacts one AT base pair. To enlarge binding site size and improve affinity, the number of N-methylpyrrole units can therefore be increased. However, for compounds containing more than six pyrroles this prediction is no longer valid since the molecule becomes out of phase with the base pairs along the minor groove floor. In fact, the pyrrole-pyrrole distance is about 20% longer than required for perfect match (Goodsell and Dickerson, 1986). In addition, compounds with five or more pyrrole rings are found to be over-bent relative to the pitch of the DNA helix resulting in decreased binding affinities for longer oligopyrroles (de Clairac et al., 1999).

To circumvent this mismatch problem, a flexible amino acid (β-alanine) can be introduced in the center of the pyrrole ring system to restore register of the recognition elements and relax the curvature of these crescent-shaped molecules.

Attempts have been made to increase the size of the binding sites of these DNA-binding molecules. For example two netropsin or two distamycin molecules have been joined together to form dimers using a variety of different linkers to achieve binding sites of 8 to 10 bases (Neamati et al., 1998, Wang and Lown, 1992). Furthermore, it has been proposed to tether together polyamides of the hairpin type using β-alanine or 5-aminovaleric acid (International patent application WO 98/45284). However, none of these proposed structures have provided satisfactory specificity.

It is an object of the present invention to provide DNA-binding molecules with high specificity and affinity for in vivo and in vitro use.

Molecules meeting this objective and which can be seen to be highly improved tandem linked DNA binding elements have been developed.

The inventors have established that the nature of the link between the DNA-binding elements (or “modules”) and the relative orientation of the linked elements are important factors in the proper functioning of each module. The characteristics which the linker must exhibit in order to achieve the above objective have been identified and are described below.

The invention thus relates to tandem linked highly sequence-specific DNA-binding molecules.

More particularly, the invention concerns a DNA binding molecule, capable of sequence specific binding to the minor groove of double-stranded DNA, characterised in that it comprises at least two sequence specific DNA-binding elements, covalently linked by an amphipathic, flexible linker molecule, at least one of said DNA-binding elements being non-proteinaceous.

In accordance with the invention, each DNA binding element alone may have relatively low specificity and affinity, but covalently linked to each other using an amphipathic, flexible linker, a compound is obtained that by far exceeds the specificity and affinity of the individual DNA binding elements.

The inventors have found that covalently linked oligopyrroles in accordance with the invention efficiently provide specificity for sequences as long as 15-18 base pairs.

The inventors have demonstrated the excellent specificity and affinity of the compounds of the invention firstly by targeting <<SARs>> (scaffold associate regions) which are candidate cis-acting regions of chromosome dynamics. The sequence hallmark of SARs are numerous AT-tracts (short motifs of A and T bases) that are generally separated by short, mixed sequence spacers, Resulting in clustered AT-tracts (Adachi et al., 1989; Bode et al., 1992; Kas and Laemmli, 1992)

This approach has also been further extended to target sequences containing all four Watson-Crick base pairs by the use of so-called tandem hairpin molecules that have little or no base degeneracy, composed of predominantly heterocyclic building blocks which are positioned opposite to each other with each unit recognizing one single base.

According to the invention, the linker which links the DNA-binding units is an amphipathic flexible linker molecule.

In the context of the invention, amphipathic means that the linker molecule has both polar and non-polar parts. The non-polar part is water-insoluble and is thus hydrophobic (or lipophilic) and soluble or miscible with non-polar solvents. The polar part is water-soluble and is thus hydrophilic.

Steps in the interaction process of a DNA minor-groove binding element involve a transfer from the aqueous solution surrounding the DNA into the hydrophobic environment of the minor groove. If the ligand is positively charged, counter ions territorially bound to the DNA will be released. In the minor groove, the element can form a variety of interactions, including hydrogen bonds and Van der Walls' interactions. Specificity of binding to a target sequence of the element in the minor groove is based on molecular complementarity of the recognition units of the moiety and the bases of the DNA target.

According to the invention, the tethering of said elements with an amphipathic, flexible linker serves to promote the bi- or multi-dentate energetically favourable interaction of the multiple elements with the DNA strand. The amphipathic nature of the linker increases the water solubility of the DNA-binding molecule. This property of the linker enables unbound or unfavourably bound elements to “escape” from the hydrophobic environment of the minor groove into the aqueous solution surrounding the DNA, to then reach DNA targets where specific energetically favourable interactions can occur.

According to the invention, the linker is necessarily at least bifunctional, i.e. it comprises at least two functional or “reactive” groups through which the link between two tandemly oriented DNA-binding elements is established. Preferably, but not necessarily, the linker is heterobifunctional, meaning that the linker molecule contains at least two different reactive groups. These groups are usually, but not always, at the extremities of the linker molecule.

Examples of suitable functional groups are amino, carboxyl, thiol, haloacetyl, aldehyde, amino-oxy, maleimide groups, a symetrical anhydride and halogen atoms. Particularly preferred are amino and carboxyl groups. In such a case, the C-terminus of the linker is bound to the N-terminus of a first DNA-binding element and the N-terminus of the linker is bound to the C-terminus of the next DNA-binding element.

The DNA-binding elements are linked in a tandem manner, i.e. consecutive DNA-binding elements are linked in the same orientation with respect to each other, for example in a head-to-tail configuration. In the case of DNA-binding elements which have amino and carboxy termini, for example pseudopeptide polyamide molecules, the amino terminus of a first DNA-binding element is tethered via the linker to the carboxy-terminus of a second DNA-binding unit. The individual DNA-binding elements are thus all oriented in the same direction, greatly facilitating the binding of the molecule to the DNA. In the context of the invention, “tandem” means in the same orientation, and “inverted” means in opposite orientation.

The DNA-binding molecule thus binds in a multidentate mode to a given strand of DNA. In other words, the DNA-binding molecules of the invention are composed of DNA-binding or elements separated by linkers which are essentially devoid of the capacity to bind the minor-groove of DNA. All the elements in a given DNA-binding molecule bind in tandem orientation to a given strand of DNA. For DNA-binding molecules having amino and carboxy termini, the binding to the DNA is normally in the “parallel” orientation, i.e. the DNA-binding molecule binds in an N→C direction parallel to the 5′→3′direction of the DNA.

The linker may or may not be involved in DNA-interactions. For example, the linker may contain positively charged groups which interact with the phosphate backbone of the DNA. The linker may also include a DNA-intercalating side group. According to a preferred variant the linker does not contain any element which has DNA-binding properties.

The linker molecules used according to the invention are preferably non-immunogenic and non-toxic and have increased resistance to proteolytic degradation. They are preferably non-self aggregating, and do not have long stretches of methylene groups, i.e. 3 or more methylene groups, thereby reducing strong van der Walls' interactions.

According to a preferred variant of the invention, the linker in the general formula (I) below, is represented by (L)ₘ wherein “m” represents an integer having a value equal to, or greater than one. In particularly preferred variants, “m” has the value 1. According to other preferred variants, “m” has a value greater than 1, for example 2 to 10, or 3 to 8, and the amphipathic linker (L)ₘ thus comprises an assembly of linker sub-units (L). In such a case, the assembled linker (L)ₘ has an overall amphipathic character, and at least one (L) su-unit is amphipathic. Preferably, more than one linker sub-unit, and most preferably all linker sub-units are individually amphipathic.

The total length of the linker (L)ₘ is generally speaking between 5 to 250 angstroms, for example 5 to 50 Angstroms. This corresponds to a length of approximately 4 to 42 interatomic bonds. The number of linker sub-units (L) can be multiplied to achieve a length corresponding to the number of DNA bases to be spanned.

Examples of suitable linkers are molecules comprising one or more polar groups such as ether groups and/or eater groups for example molecules derived from ethylene oxide or propylene oxide, Derivatives of ethylene oxide (CH₂CH₂O) are particularly preferred, for example oligomers of ethylene oxide having functional groups at the extremities. Such derivatives are schematically represented by the following structure:

F¹—[CH₂CH₂O]ₙ—F²

where F¹ and F² represent any functional groups, for example those listed above, and may be the same or different, and “n” may have a value from 1 to 20, for example 1 to 10, or 1 to 5.

Oligoglycine (NH—CH₂—CO)ₙ can also be used as an amphipathic linker of the invention. A particularly preferred example is a linker comprising one or more units of 8-amino-3, 6-dioxaoctanoic acid (Ao).

The linker may also contain residues which are not directly involved in linking, for example residues for chain conversion such as glutamic acid or succinic anhydride.

At least one, and preferably all of the DNA-binding elements of the molecule of the invention are non-proteinaceous. In the context of the invention, “non-proteinaceous” means that a given DNA-binding element is composed, preferably but not necessarily exclusively, of non-naturally-occurring amino acids. Non-naturally-occurring amino acids are amino-acids other than those used by living cells to make proteins, for example organic heterocyclic amino acids such as pyrrole, imidazole, triazole etc.

The DNA-binding molecule of the invention thus comprises a plurality of DNA-binding elements linked to each other with an amphipathic linker. According to a preferred embodiment, at least one of the DNA-binding elements of the molecule of the invention comprises an oligomer containing one or more organic heterocyclic amino acid residues. Such molecules are known as “polyamide” DNA-binding molecules, or “pseudopeptides”.

Particularly preferred organic heterocyclic amino acid residues are those having at least one annular nitrogen, sulphur or oxygen, such as pyrrole, imidazole, triazole, pyrazole, furan, thiazole, thiophene, oxazole, pyridine. The organic heterocyclic residues may also be derivatives of any of these compounds wherein one or more of the heteroatoms are substituted by a substituent which is DNA-binding or non-DNA-binding. Examples of DNA-binding substituents are pyrrole, imidazole etc as listed above.

According to a particularly preferred embodiment, at least one DNA-binding oligomer includes heterocyclic residues chosen from N-methylpyrrole (Py) and/or 3-hydroxy N-methylpyrrole (HP) and/or N-methylimidazole (Im).

The DNA-binding element may further comprises at least one, for example 2, 3 or 4 aliphatic amino acid residue such as a β-alanine (β) residue or a 5-aminovaleric acid residue. β-alanine is particularly preferred.

In a further preferred variant, the DNA-binding molecule of the invention has the general formula (I):

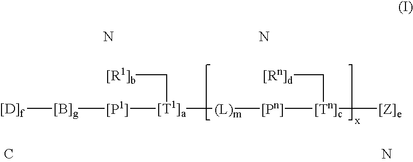

wherein

each of P¹ to Pⁿ represents a DNA-binding element, said element comprising multiple organic heterocyclic or aliphatic residues or fluorescent derivatives thereof;

each of R¹ to Rⁿ represents a DNA-binding element, said element comprising multiple organic heterocyclic or aliphatic residues or fluorescent derivatives thereof;

x represents an integer from 1 to 20, with the proviso that when x is greater than 1, the multiple copies of [Rⁿ], [Lⁿ], [Pⁿ] and [Tⁿ] may be the same or different, and may be the same or different from [R¹], [P¹] and [T¹];

[T] represents a multifunctional linking molecule providing a covalent link between DNA-binding elements [R] and [P], with the proviso that if “e” represents 0, [T^{x+1}] can be bifunctional;

n is an integer equal to (x+1);

each of a and c independently represent 0 or 1

each of b and d independently represent 0 or 1. with the proviso that when a represents 0, b also represents 0, and when c represents 0, d also represents 0,

[D] represents an end group or an effector moiety

[L]ₙ represents an amphipathic, flexible linker molecule linking the DNA-binding elements in a tandem orientation with respect to each other;

m represents an integer from 1 to 10,

B represents a spacer unit such as β-alanine,

[Z] represents an end group or an effector moiety;

each of f, g and e independently represent 0 or 1,

each solid line represents a covalent bond

N and C indicate the N- and C-terminal extremities of the molecule, respectively.

In the above formula I, the DNA-binding elements are represented by [R¹], [P¹], [R²] and [P²]. When [T¹] and/or [Tⁿ] is present, the covalently linked unit of [R¹], [P¹] and [T¹] is considered as a DNA-binding unit, and [Rⁿ], [Pⁿ] and [Tⁿ] is also a DNA-binding unit.

In the formulae of the present inventions an element represented in square brackets with a sub-script outside the square brackets, for example “[R]_{b}”, indicates multiple copies of the element, which, unless otherwise indicated, may be the same as each other or different from each other, the number of multiples being equal to the value of the subscript. An element represented in square brackets with a super-script inside the square brackets, for example “[Rⁿ]”, indicates the “n^{th}” copy of that element, the first to the n^{th} copy being the same as each other or different from each other.

The DNA-binding elements [P] and [R] in Formula (I) preferably comprise heterocyclic residues chosen from pyrrole, imidazole, triazole, pyrazole, furan, thiazole, thiophene, oxazole, pyridine, or derivatives of any of these compounds wherein one or more of the heteroatoms is substituted. The substituents may be DNA-binding or non-DNA binding.

In a particularly preferred embodiment, a, b, c and d in Formula (I) represent <<0>>, that is the [T] and [R] moieties are absent. Such a, molecule will be referred to herein as a <<linear>> DNA-binding molecule. Generally such linear molecules have the general formula (II):

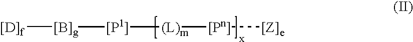

wherein [P1], [Pn], [L], [D], [Z], x, m, f, g and e have the previously defined meanings

and a dotted line represents a covalent bond which can be present or absent.

“Linear” polyamides, as referred to herein, are polyamides composed of a single N→C strand of amino acid residues. Such linear molecules can bind DNA, either as a single molecule in a 1:1 binding mode, or in a 2:1 binding mode, wherein two linear molecules align in an anti-parallel manner in the minor groove, forming binding pairs between the residues of the first molecule and those of the second molecule.

In Formula (II), each of the the DNA-binding elements [P¹] to [Pⁿ] preferably independently have the general formula (III)

—[U¹-[U]ₛ]— (III)

wherein:

each U is a monomeric unit chosen from a heterocyclic amino acid residue, or an aliphatic amino acid residue or a fluorescent derivative thereof, and

s is an integer from 1 to 15, preferably from 2 to 8, and a dotted line represents a covalent bound which may be present or absent.

The linear DNA-binding molecules of the invention preferably have at least one [U] moiety chosen from N-methylpyrrole (Py) and/or 3-hydroxy N-methylpyrrole (HP) and/or N-methylimidazole (Im).

Furthermore, they may also contain at least one β-alanine (β) residue, or a 5-aminovaleric acid residue.

In Formula (III), the value of S is preferably from 2 to 8, for example 2 to 6, or 3 to 4.

At least one of the elements [P¹] to [Pⁿ] of the linear DNA-binding molecules may comprises between 3 to 5 heterocyclic amino acid residues, for example 4. Of these, two or more may be contiguous, for example three, four or five contiguous heterocyclic amino acid residues. Preferably, stretches of three to five contiguous heterocyclic amino acid residues are separated from each other by a β-alanine residue.

Particularly preferred linear molecules comprise at least one [P¹] to [Pⁿ] element having the formula (IV):

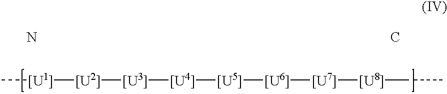

wherein U is as previously defined,

[U₄] is β-alanine,

[U₁] to [U₃], and [U⁵] to [U₇] are chosen from N-methylpyrrole

(Py) and/or N-methylimidazole (Im),

[U₈] may be present or absent, and if present is preferentially β-alanine,

and a dotted line represents a covalent bound which may be present or absent.

In Formula (IV) [U¹] to [U³], and [U⁵] to [U⁷] may each be N-methylpyrrole (Py).

In another preferred embodiment at least one of [P1] to [Pn] of the DNA-binding molecule has the fomula (V):

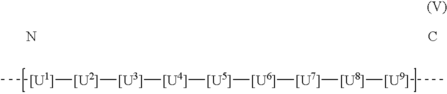

wherein:

U is as previously defined,

[U₁] to [U₈] are chosen from N-methylpyrrole (Py), N-methylimidazole (Im) and a β alanine residue,

with the proviso that the [U] immediately adjacent, on the N-terminal side, to each Im is a β alanine residue, [U₉] may be present or absent, and if present is preferentially β-alanine, and a dotted line represents a covalent bound which may be present or absent.

An example of such a [P¹] to [Pⁿ] element has the formula (VI):

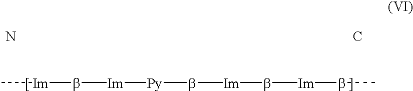

According to a preferred embodiment, the number of repeat x DNA elements contained within a linear molecule, i.e. the value of <<x>> in Formula (II) is from 2 to 10, for example 2, 3, 4, 5, 6, 7, 8, 9, or 10.

In linear molecules of Formula I, the DNA-binding links [P¹] and [Pⁿ] are linked in the same molecular orientation (i.e. in tandem) by the linker L.

In addition to the linear molecules, the invention also relates to branched molecules, for example <<hairpin>> molecules. Such branched molecules generally have the general formula (VII)

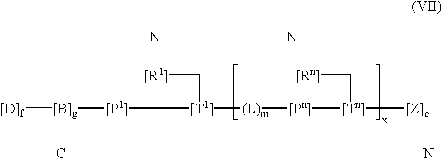

wherein [R¹], [P¹], [Rⁿ], [Pⁿ], [T¹], [Tⁿ], (L), [D], [B], [Z], m, n, g, f and e have the previously defined meanings.

In Fomula (VII), each of the DNA-binding elements [P1] to [Pn] and [R1] to [Rn] may independently have the formula (VIII)

—[U1-[U]s]— (VIII)

wherein:

each U is a monomeric unit chosen from a heterocyclic amino acid residue, or an aliphatic amino acid residue or a fluorescent derivative of the foregoing, and

s is an integer from 0 to 15, preferably from 1 to 6, and a dotted line represents a covalent bound which may be present or absent.

The branched molecules such as hairpin polyamides, preferably contain at least one heterocyclic amino acid residue comprising an annular nitrogen. More specifically, at least one of [P¹] to [Pⁿ] or [R¹] to [Rⁿ] preferably contains a residue of N-methylpyrrole (Py) and/or 3-hydroxy N-methylpyrrole (HP) and/or N-methylimidazole (Im). [P¹] to [Pⁿ] or [R¹] to [Rⁿ] advantageously further contain an aliphatic amino-acid residue such as a β-alanine (β) residue

In Formula VIII, “s” is an integer from 0 to 15, preferably 1 to 6, for example, 3, 4 or 5.

The branched molecules of Formula VII comprise a moiety [T] which serves to covalently link the upper DNA-binding element [R] with the lower DNA-binding element [P]. [T] may be any molecule suitable for providing this link, and may have DNA-binding properties or not, [T] may be positioned between any residues in the upper strand and lower strand. [T] is at least bifunctional in order to allow the linkage of the two strands of the molecule. [T] may however also have more functional groups, being for example trifunctional. This allows addition of any further moieties, such as effector moieties, if desired at this site. The functional groups of [T] are for example, chosen from amino, carboxyl, thiol, haloacetyl, aldehyde, amino-oxy, maleimide groups, a symmetrical anhydride and halogen atoms, but can also include any other suitable groups.

A preferred example of [T] is a “turn” molecule derived from an amino acid, giving rise to a “U” shaped molecule, such as a hairpin polyamide. According to this variant, [T] is chosen for example, from γ-aminobutyric acid or diaminobutyric acid or an amino acid with a side group, or any other molecule having at least 3 reactive groups, or a fluorescent derivative of the foregoing,. If “e” in Formula VII represents 0, [T^{x+1}] can be bifunctional, for example γ-butyric acid. Other suitable [T] linkers include “H” pins.

According to the hairpin variant of the invention, a first DNA-binding unit composed of [P¹], [T¹] and [R¹], is linked in tandem via the linker to a second DNA-binding unit composed of [Pⁿ], [Tⁿ], and [Rⁿ].

At least one of the elements [P¹] to [Pⁿ] of the hairpin DNA-binding molecules may comprise between 3 to 5 heterocyclic amino acid residues, for example 4. Of these, two or more may be contiguous, for example three, four or five contiguous heterocyclic amino acid residues. Preferably, stretches of three to five contiguous heterocyclic amino acid residues are separated from each other by a β-alanine residue.

The invention also concerns hairpin DNA-binding molecule wherein at least one [Pⁿ] element has the formula (IX):

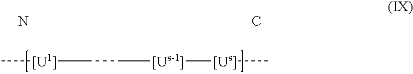

and at least one [Rⁿ] element has the formula (X):

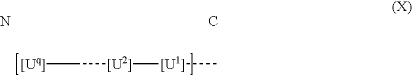

wherein each U represents independently N-methylpyrrole (Py),or 3-hydroxy N-methylpyrrole (HP), or N-methylimidazole (Im) or N-methyl pyrazole (Pz), or 3-pyrazolecarboxylic acid (3-Pz), or β-alanine (β), q and s are independently integers from 1 to 10,

and a dotted line represents a covalent bond which can be present or absent,

wherein the U residues of [Pⁿ] form anti-parallel pairs with the U residues of [Rⁿ]:

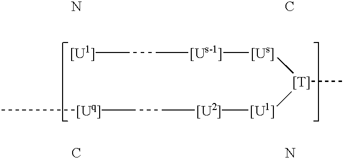

said pairs being chosen from Py/Im, Im/Py, Py/Py, Hp/Py, Py/Hp, β/Py, Py/β. β/Im, Im/β, Im/Im, Pz/Py, 3-Pz/Pz, and β/β.

In the formulae (I), (II) and (VII), [Z] may be any end group or an effector moiety, for example any conjugated chemical group such as an affinity tag, a fluorescent label, a peptide, a reactive group, or a toxin. The DNA-binding molecules can therefore be used to target effector molecules intracellularly.

Similarly, in the above Formulae, [D] represents an end group such as dimethylaminopropylamide, 3-aminopropylamine-N-methyl N-propylamide, or a fluorescent derivative thereof. Alternatively, [D] may comprise an effector moiety.

Indeed, according to a particularly preferred variant of the invention, the DNA-binding molecules comprise an effector moiety. In view of the excellent affinity and specificity which these cell-permeable molecules show for their DNA targets, they can be used to deliver a large number of different types of compounds to the nucleic acids and cellular compartments in question.

The “effector moiety” is any chemical group or molecule which mediates a ;unction other than, or in addition to, sequence-specific recognition of DNA in the minor groove. For example, the effector moiety may be a peptide, a fluorescent label, a reactive group, a toxin or an affinity tag.

The effector moiety can be linked to the molecule at any suitable site, preferably by a covalent bond, for example to any of the heterocyclic or aliphatic amino acid residues, or to the carboxy or amino termini, or to the [T] or [L] moieties. In formulae (I) and (VII) particularly preferred sites for linkage of the effector moiety are represented by [D] and/or [Z]. Other particularly preferred site for linkage of an effector moiety is linkage to a pyrrole residue.

The effector moiety is capable of carrying out at least one of the following functions: visual detection, nucleic acid cleavage, binding to the major groove of nucleic acid, inhibition of binding to the major groove of nucleic acid, protein binding, inhibition of protein binding, chemical modification of DNA, distortion of DNA structure.

Particularly preferred effector moieties include a fluorescent moiety, an alkylating moiety, an intercalating moiety, nucleotides and derivatives thereof, or combinations of any of the foregoing. As particular examples, one can cite antisense oligonucleotides or ribozymes, isothiazolone derivatives; acridine or derivatives thereof; porphyrins; cisplatin or derivatives thereof; anthracyclins or derivatives thereof. Illustrative embodiments of effector moieties are indicated in the examples below.

The invention also relates to mixed linear and hairpin molecules in which at least one DNA-binding sub-units is linear and at least one is hairpin. In the general formula (I), these molecules have at least one DNA-binding element containing [T], [R] and [P] moieties, and at least one DNA binding element which is free of [T] and [R] moieties.

The multiple [R] and [P] elements of the molecules, whether linear, hairpin or mixed, may all be identical, or alternatively may differ in length and/or composition.

According to a particular preferred embodiment, DNA-binding molecules of formula I have “x” equal to 1, 2, 3, 4 or 5, “s” equal to 3 or 4, “n” equal to 2 or 3, “e” equal to 1 or 0, “g” equal to 1 or 0 and “f” equal to 1 or 0. Molecules having x equal to 1 are particularly preferred. Such molecules are dimers, and may be homo- or heterodimers.

The molecules of the invention have exceptional DNA-sequence specificity. Preferably, they have the capacity to bind in a sequence specific manner to a DNA recognition sequence of at least 6, preferably at least 10 and most preferably at least 14 base pairs in length. In the context of the invention, sequence specificity in vivo means that the normal functions of the cell other than those mediated by the targeted sequence, are not perturbed by the molecule. The molecule therefore acts on its target without causing effects which the cell could not tolerate, over and above the sought effect.

A further advantage of the molecules of the invention is that they are small, that is they preferably have a molecular weight no greater than approximately 8 kDa for example less than 6 kDa or less than 5 kDa, particularly between 1 kDa and 5 kDa. These molecules are cell-permeable, greatly facilitating their administration as drugs etc. The cell-permeability is usually conserved even when one or more effector moieties are included in the DNA-binding molecules. As the size of the molecules increases, permeability may become less, and it is therefore advantageous to carry out any necessary chemical modification of the compound to conserve or restore cell permeability. This can be done for example by chemical modification of one or more of the heterocyclic amino acid residues. Cell permeability and/or solubility of the compound can be modulated in this manner. The chemical modification typically comprises the addition of a polar side chain, for example a propylamine side chain, or a bulky side chain to a pyrrole residue.

A further modification which could be made to enhance permeability and/or solubility is the addition of a charged amino acid such as Histidine, Arginine, Lysine.

A particular advantage of the DNA-binding compounds of the invention, resulting from the use of the amphipathic linker, particularly the derivatives of ethylene oxide, is the enhanced solubility of the compounds in aqueous media compared to polyamide multimers containing hydrophobic linkers. Indeed, the amphipathic nature of the linker confers a degree of hydrophilic character on the molecule, giving rise to an adequate solubility in aqueous solutions such as cell culture media or physiological solutions. The tandem-linked molecules of the invention do not precipitate out (i.e., do not form crystals) in cell culture, in contrast to multimers linked with conventional hydrophobic linkers such as 5-amino valeric acid. It has been demonstrated by the inventors that the molecules of the invention conserve solubility even after addition of a hydrophobic effector moiety such as an alkylating group (e.g. chlorambucil). This characteristic facilitates use of the linked polyamides as agents for delivery of effector moieties to intracellular compartments. The solubility of the compounds of the invention can be verified using the assay indicated in Example 10 below.

The DNA-binding molecules of the invention also exhibit exceptional binding affinity for example, an apparent binding affinity of at least 5×10⁷M⁻¹, preferably at least 1×10⁹M⁻¹ and most preferably at least 5×10¹⁰M^{-1.}

The invention also relates to a process for binding double-stranded DNA in a sequence-specific manner, comprising contacting a DNA-target sequence within said DNA with a DNA-binding molecule according to the invention, in conditions allowing said binding to occur. The molecules used in this process may be hairpin, linear or mixed.

The process may be carried out in vivo, in vitro or ex vivo. In vivo processes are particularly preferred.

When the process of binding is carried out in a cell, the cell may be eukaryotic or prokaryotic. Eucaryotic cells are particularly preferred, for example vertebrate cells, an invertebrate cells, plant cells, mammalian cells, insect cells, or yeast cells.

The double stranded target DNA may be endogenous to the cell or it may be heterologous to said cell.

The target is preferably a chromatin element, for example a SAR-like sequence, or a GAGAA repeat sequence.

For intracellular use, the target sequence preferably has at least 6 or 8 and preferably at least 10 or at least 12 or 15 bases. High specificity is thus achieved within the cell.

The target sequence is preferably a cis- or trans-acting element mediating chromosome function. Use of the tandem-linked molecules of the invention to target such a sequence gives rise to cis- and/or trans-regulation of chromosome function.

The double stranded DNA target sequence may also comprises a site mediating the activity of one or more regulatory factors, for example transcription regulatory factors, DNA replication factors, factors for enzymatic activity, or factors involved in chromosome stability.

DNA-binding molecules of the invention can be designed to target many DNA sequence using the pairing rules known in the art. Table 1 below provides examples of the binding preferences of frequently used residues. Sequence-specific effects normally influence the precise binding behaviour of some heterocycles. Table 1 therefore provide general guidelines which can be adapted, if necessary, to fit particular situations.

Table 2 shows residue pairs which can be substituted for other pairs.

The composition of the DNA-binding molecule is chosen as a function of the sequence of the targeted DNA, on the basis of pairing rules known in the art, for example as indicated in Tables 1 and 2. For linked polyamides, particularly hairpins, containing a number ‘n’ of amino acid residues, the target sequence usually comprises n+3 bases.

TABLE 1
Guidelines for binding preferences

| Residue or | |
|---|---|
| Pair of residues | DNA binding preference |
| Im/Py | G—C |
| Py/Im | C—G |
| Py/Py | A—T and T—A |
| Hp/Py | T—A |
| Py/Hp | A—T |
| β (preceded on C- | A—T or T—A |
| terminal side by Dp) | (flanking core sequence) |
| Pz/Py | A—T or T—A |
| 3-Pz/Py | G—C |
| β/β | T—A or A—T |
| β/Py | T—A or A—T |
| Py/β | T—A or A—T |
| Im/β | G—C |
| β/Im | C—G |
| Unpaired Im (internal, | G or C, but tolerated by W's, |
| not N-terminal, in a | particularly if preceded by an N- |
| single-stranded molecule) | terminal pyrrole, but less well if |
| | preceded (N-terminal) by a β |
| Dp (C-terminal) | W |
| Unpaired Py | A—T or T—A |
| Unpaired Hp | A—T |
| Unpaired Im (in unpaired | Preferably G or C |
| overhang of a linked | |
| molecule of invention) | |
| γ | Optimally positioned on a W |
| Ethylene oxide linker | Optimally bridges W (but can loop |
| | out, opposing no nucleotide) |

TABLE 2
Substitution of binding pairs by Hp-containing pairs

| Residue or | Possible |
|---|---|
| Pair of residues | Substitutions |
| Im/β | Im/Py |
| β/Im | Py/Im |
| Py/β | Hp/Py or Py/Hp |
| β/Py | Hp/Py or Py/Hp |
| Hp/β | Hp/Py |
| β/Hp | Py/Hp |
| β/β | Hp/Py or Py/Hp |

Legend of Tables 1 and 2:

Im: N-methyl imidazole

Py: N-methyl pyrrole

Hp: N-methyl hydroxypyrrole

Dp: C-terminal dimethylaminopropylamide

β: β-alanine

Pz: N-methyl pyrazole

3-Pz: 3-pyrazolecarboxylic acid

γ: γ-aminobutyric acid, (or diaminobutyric acid)

W: A or T

The invention also relates to a process for modulating chromosome function in a eukaryotic cell, comprising the step of contacting a genomic DNA element comprising a binding site mediating chromosome function, with a tandem-linked DNA-binding molecule of the invention and having the capacity to bind in a sequence-specific manner to said element, said step of contacting being carried out in conditions permitting binding of said compound to said element, wherein the binding modulates chromosome function.

The invention further relates to a process for modulating the function of a DNA element in a eukaryotic cell, comprising the step of contacting a genomic DNA element, so-called <<chromatin responsive element>> (CRE), with a tandem-linked DNA-binding molecule of the invention and having the capacity to bind in a sequence-specific manner to said CRE, said step of contacting being carried out in conditions permitting chromatin remodelling of the CRE by said compound, wherein said chromatin remodelling of the CRE alters the activity of one or more other DNA elements, so called <<modulated DNA elements>> in the genome.

Non-human organisms comprising the cells of the invention are also comprised within the invention, for example a non-human animal, which may be a transgenic, non-human animal, or a plant including a transgenic plant.

The invention also relates to a pharmaceutical composition comprising a DNA-binding compound of the invention in association with a physiologically acceptable excipient, carrier, adjuvant, stabilizer or vehicle. The composition may be administered orally, sub-cutaneously, topically, rectally, intravenously, intramuscularly or by inhalation spray.

The compounds and compositions of the invention may be used in therapy, particularly in the treatment of disorders of genetic origin.

The compounds and compositions of the invention may be fluorescent or fluorescently labelled. The fluorescent label may be a fluorescent dye such as fluorescein, dansyl, Texas red, isosulfan blue, ethyl red, malachite green, rhodamine and cyanine dyes.

The fluorescent compounds can be used for probing the epigenetic state and location of DNA in chromosomes and nuclei, for chromosome visualisation and marking in diagnosis, forensic studies, affiliation studies, or animal husbandry.

### FIGURE LEGENDS

FIG. 1: Chemical structure and the oligopyrrole monomers and dimers.

The structures of the dimers Lex18 and Lex10 are shown. Both dimes are composed of the same oligopyrroles monomers (P7 and P9) joint by either a short (Lex18) or a long (Lex10) linker. The linker of Lex10 contains three ethylene oxide spacer amino acids (AO) and Lex18 only one. The flexible linker allows bidentate binding of both oligopyrrole moieties to long or bipartite AT-tracts of 15-18 bases. Amino- and carboxyl termini are marked with N and C respectively.

FIG. 2: DNase I footprint assays with P9, P7, P13, Lex9 and Lex10.

DNase I cleavage pattern in the presence of P9, P7, P13, and dimers Lex9 and Lex10. . Ligand concentrations are indicated at the top of each lane. The position of each of the AT-tracts is indicated by square brackets. Panel A shows the footprints of monomers P9 and P7 on probe W9. This probe is composed of head-to-tail tandem repeats of an oligonucleotide, with a 9 bp AT-tract. Panel B shows the footprint of P13 on a probe with one single W9 insert at the indicated position. Panel C shows the DNase I cleavage pattern of the same probe as in panel B in the presence of Lex9 and Lex10. Ligand concentrations are again indicated at the top of each lane (in nM). The position of each of the AT-tracts is indicated by square brackets, Kₐₚₚs (apparent dissociation constants) are listed in Table (3). Note that P13 (not dimers Lex9 and 10) was found to be very GC-tolerant since its footprint expanded rapidly at increasing ligand concentration from W9 into the flanking mixed sequences to eventually protect (coating) the entire probe.

FIG. 3: Binding of Lex10 and Lex18 to SAR

Panel A: DNase I cleavage pattern of end-labeled SAR probe in the presence of Lex10. Ligand concentrations (nM) are indicated at the top of each lane. The position of each of the AT-tracts is indicated by square brackets. Panel B shows the affinity cleavage reaction by Lex18E on the SAR probe (same probe as in panel A). Panel C: DNAse I footprinting experiment with P31 and affinity cleavage with P31E are shown on GAF31 and the Brown I probes. The GAF31 probe contains a (AAGAG)₂ motif and GAGA factor (GAF) binding site from the Ubx promoter (Biggin et al., 1988). Note that P31 does not bind the typical GAF binding (Ubx). The Brown I oligo (a tandem repeat) includes an (AAGAG)₅ binding site and a degenerate P31 binding site (AACAC)₂ as indicated. P31 concentrations used (nM) are indicated. Lanes labeled P31E (top) are affinity cleavage reactions with 1 nM of P31E on either probe. Binding orientations of P31E on these probes are indicated by arrowheads on the brackets pointing towards the N-terminus of the molecule. The letter G refers the G nucleotide cleavage reaction. Panel D shows the sequence of this SAR probe and the positions of the major AT-tracts. Protected region are indicated with boxes. The vertical arrows reflect the affinity cleavage site and approximate strength. Panel E shows a binding model of dimers Lex10 and Lex18 on the W17 tract (see panel C) of the SAR probe (top).

FIG. 4: Staining of Drosophila nuclei and polytene chromosomes with fluorescently tagged oligopyrroles.

Isolated Kc nuclei were stained with ethidium bromide and fluorescein-tagged oligopyrroles as indicated. Note that P9 (panel A) and Lex9F (panel B) highlights as intense green foci satellites I and III and that %the general nucleoplasmic staining of P9 is more pronounced with than that of dimer Lex9P. This is best seen in the gray scale insert (panel C) where the total DNA signal (EB) and the Lex9F or P9F signals are shown separately. Note that the nuclear subregion stained intensely with ethidium bromide represents the nucleolus. Panel D shows a single polytene chromosome stained with ethidium bromide (red) and Lex 9F. The two major signals of Lex9F abutting the chromocenter on chromosome IV and IIIR represent satellite I (indicated). Other important Lex9F signals appearing yellow are in the arm of chromosome 4 and within the chromocenter. This latter signal may represent the under replicated SAR-like sequence satellite III (indicated). Panel E shows the transverse striations of the Lex9F in green (overlap yellow) which are thought to reflect the positions of SARs along the euchromatic arms of polytene chromosomes. The red signal of ethidium bromide shows the classic banding pattern. For panel E, colors were not blended additively as above but by using color priority where the pixel values of higher priority wavelengths are subtracted from the lower priority wavelength. This reduces color mixing, rendering the more subtle variation of green and red more visual. Micrographs were recorded on a DeltaVision epifluorescence microscopy system.

FIG. 5: Binding specificity of P31 and GAGA factor

Panel A: DNAse I footprinting experiment with P31 and affinity cleavage with P31E are shown on GAF31 and the Brown I probes. The GAF31 and Brown I probes contains a (AAGAG)₂ motif and GAGA factor (GAF) binding site from the Ubx promoter (Biggin et al., 1988). Note that P31 does not bind the typical GAF binding (Ubx). The Brown I oligo (a tandem repeat) includes an (AAGAG)₅ binding site and a degenerate P31 binding site (AACAC)₂ as indicated. P31 concentrations used (nM) are indicated. Lanes labeled P31E (top) are affinity cleavage reactions with 1 nM of P31E on either probe. Binding orientations of P31E on these probes are indicated by arrowheads on the brackets pointing towards the N-terminus of the molecule. The letter G refers the G nucleotide cleavage reaction. Panel B: DNAse I footprinting experiment with purified GAGA factor (GAF) on the GAF31 probe. Note that GAF binds both the (AAGAG)₂ motif and the binding site from the Ubx promoter.

FIG. 6: The fluorescent polyamide P31T specifically highlights the GAGAA satellite V

Isolated Kc nuclei and polytene chromosomes were stained with DAPI (blue), P31T (Texas red-labeled P31), Lex9F (Fluorescein tagged Lex9). Panel A: The green P9F foci are proposed to highlight satellites I and III. P31T marks the separate positions of the GAGAA satellites. Panels B & C: The black and white panels display the red and green channels of panel A, respectively. Panel D: Staining of brown-dominant polytene chromosome with DAPI, P31T and Lex9F. The polytene banding pattern is shown in blue (DAPI). P31T highlights in red the heterochromatic GAGAA repeats of the allele bw^{D} at 59E. Lex9F (green) highlights in polytene chromosome the position of satellite I at the base of chromosomes 4 and 3R abutting the chromocenter (FIG. 5).

FIG. 7: Oligopyrrole monomers induced chromatin opening of satellite III.

Kc nuclei were incubated with mitotic Xenopus egg extracts in the presence of the various polyamides-and then further treated with VM26 to accumulate the so-called cleavable complexes of topoisomerase II. Cleavage in Drosophila satellite III was revealed by southern blotting. Satellite III contains a major topoisomerase II cleavage site once per 359-bp repeat. The extent of the cleavage activity is reflected by the development of the ladder of multimers of the basic repeat. All panels included controls with (+) and lanes without Vm26 (−). Panel A shows the massive activation of cleavage (chromatin opening) mediated by P9 and the reduced activity P31 in this assay Panel B In contrast to monomer P9 no cleavage stimulation but abrupt inhibition is observed with Lex10. A much reduced cleavage stimulation is also observed with Lex9. Panel C demonstrates that the general fragmentation of the genome by topoisomerase II is not inhibited by, Lex10 and Lex9. DNA was separated by pulse-field electrophoresis and then probed with total Kc DNA under conditions that suppress hybridization to repeat DNA. Duplicate samples were loaded.

FIG. 8: Specific inhibition of chromosome assembly by Lex10

Panel A: The effect of Lex9 and Lex10 on the condensation of sperm nuclei to chomatids was studied in mitotic Xenopus egg extracts. Representative micrographs of the assembly products stained with ethidium bromide are shown. Ligand concentrations are as indicated.

Panel B: Condensation was inhibited by Lex10 (1 μM) or monomer P9 (2 μM). Competing oligonucleotides where then added to evaluate the specificity of the inhibition. The condensation block by Lex10 could be reversed by the addition of SAR oligo but not with the W9 or GAGA oligo which bind Lex10 poorly (doses are indicated in ng). In contrast, the P9 mediated inhibition appears non-specific and could not be reversed by an excess of competitor oligonucleotide W9.

FIG. 9: Structure of compounds P49, P50 and P51

FIG. 10:

Binding affinity (Kₐ) of linked oligopyrroles (monomer, dimer, trimer) versus binding site size.. In the top panel, it can be observed that the oligopyrrole trimer P49, designed to bind −18 Ws laws (A or T base pairs) has maximum binding affinity on W18. Specificity for these sequences is due to the much lower binding strength on shorter AT-tracts. For example, the binding affinity to P49 on W9 (K_{d}=150 nM) is ˜300 fold lower than on W18 (K_{d}=0, 75 nM).

FIG. 11:

Structure of compound P52, This compound is designed to bind the 10 bp sequence 5′-GGTTAGGTTA-3′. A single base pair insertion or deletion in the middle of this sequence was shown to abolish binding.

FIG. 12:

DNAse footprinting experiment of P52 for 5′-GGTTAGGTTA-3′.

FIG. 13:

The structures of differently linked tandem hairpin polyamides, conjugated with a hydrophobic effector moiety (chlorambucil).

FIG. 14:

HPLC chromatograms showing superposed profiles for soluble and insoluble fractions (supernatant and pellet respectively). Panel (A) shows the profiles for the valeric acid linked tandem hairpin (FIG. 13, bottom). Panel (B) shows the profile for the tandem hairpin with the amphipathic linker of the invention (FIG. 13, top). The more hydrophilic compound (panel B) also eluted earlier during the same HPLC gradient.

### EXAMPLES

Materials and methods employed in the following examples are indicated collectively in Example 11 below.

### Example 1.

Synthesis of oligopyrroles for targeting AT-tracts

To explore the biological potential of polyamides, compounds that target DNA satellite I, III, V and the interspersed SAR elements were synthesized. Satellite I (1.672 density) consists of AATAT units encompassing about 6 megabases (Mb) Satellite V (1.705 density) is composed of AAGAG repeats amounting to about 7 Mb (Lohe et al., 1993). Satellite III (1.688 density) has a much longer repeating unit (359 bp) and covers about 10 Mb (Hsieh and Brutlag, 1979). Satellite III repeats behave operationally like SARs (Kas and Laemmli, 1992), the sequence hallmarks of which are (numerous clustered AT-tracts. For example, the SAR associated with the Drosophila histone gene cluster is defined by a 656 bp EcoRl/Hinf1 fragment containing 26 AT-tracts of 8 or more Ws (A or T bases) with an average length of 10 base pairs (Gasser and Laemmli, 1986; Mirkovitch et al., 1984). Twenty of these AT-tracts are clustered and separated by a spacer of only a few nucleotides (average 4.5) of mixed base pair sequence.

The minor groove of AT-tracts can be targeted by the naturally occurring antibiotics distamycin A and netropsin, as well as by synthetic molecules that contain the same N-methylpyrrole carboxyamide ring system. These crescent-shaped molecules are bound in the center of the minor groove allowing the formation of bifurcated hydrogen bonds with the adenine N3 and thymine O2 atoms on the floor of the minor groove (Geierstanger et al., 1994).

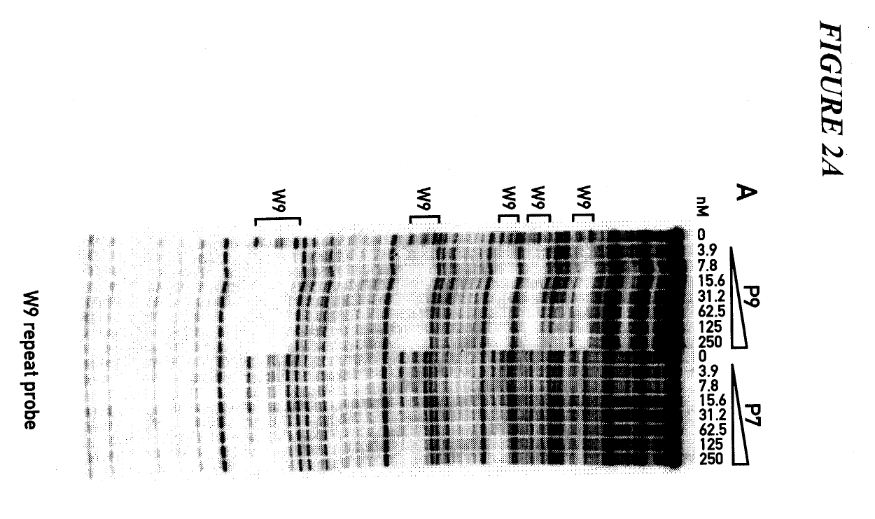

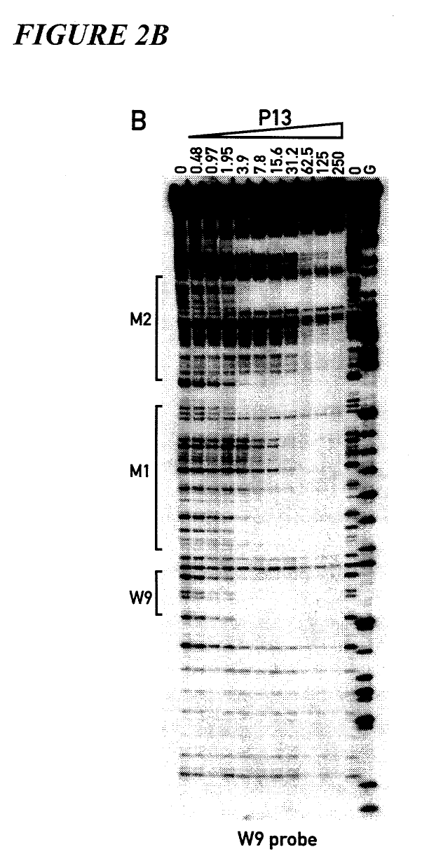

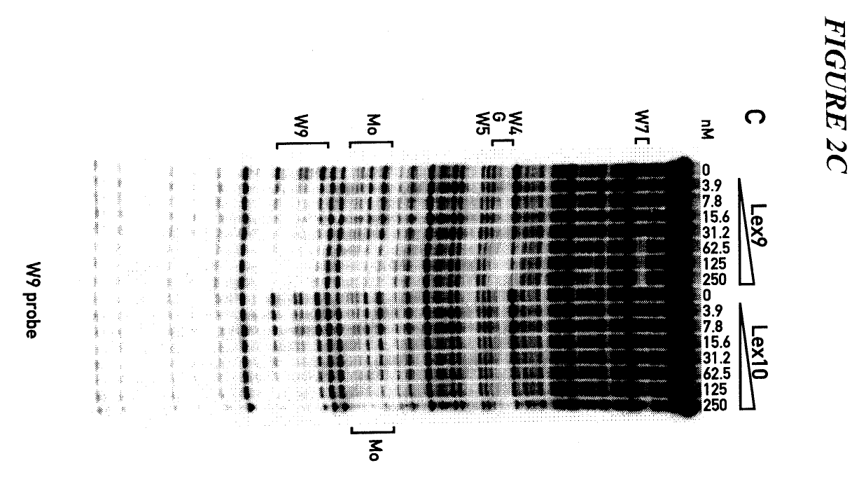

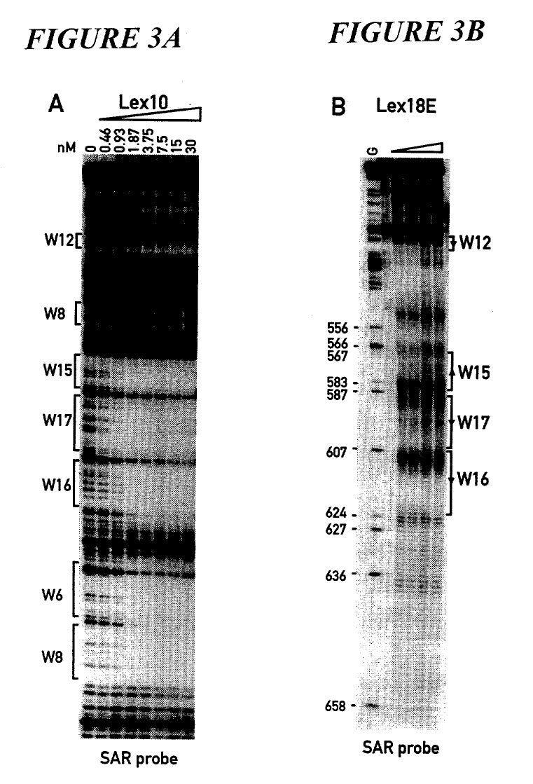

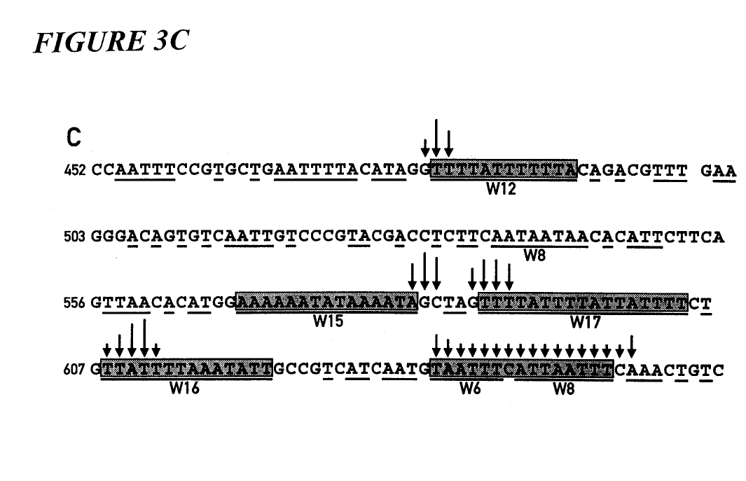

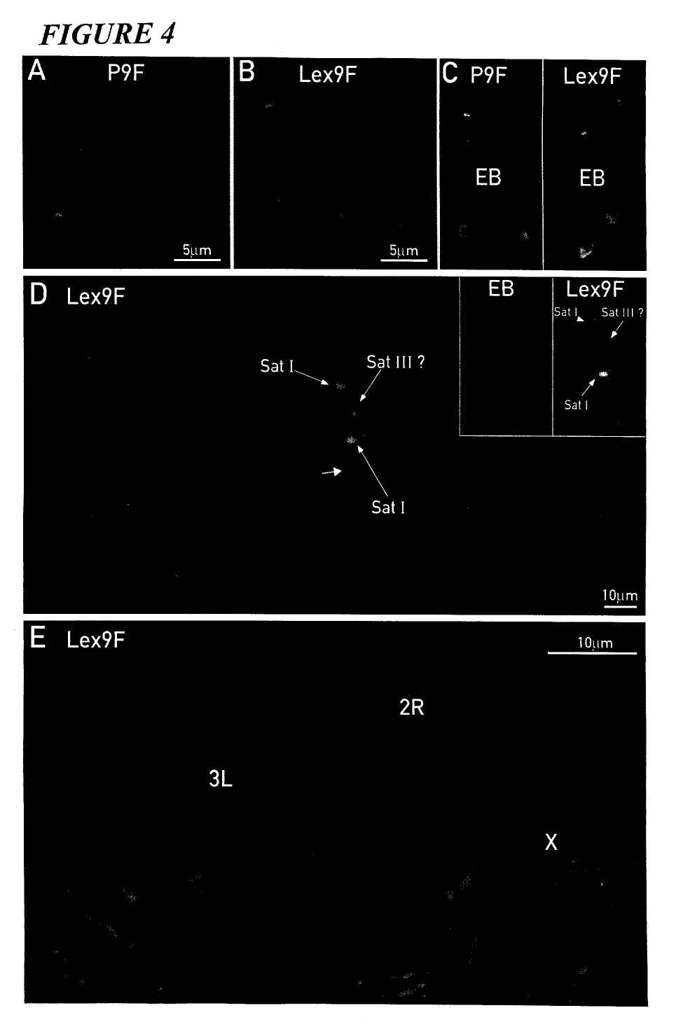

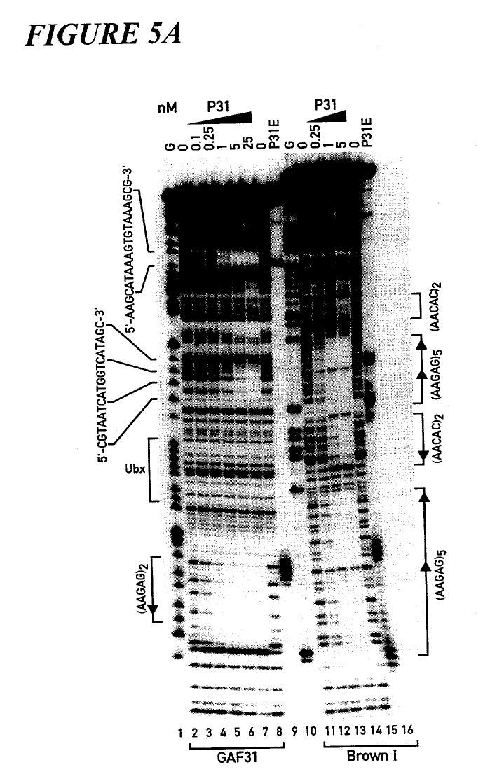

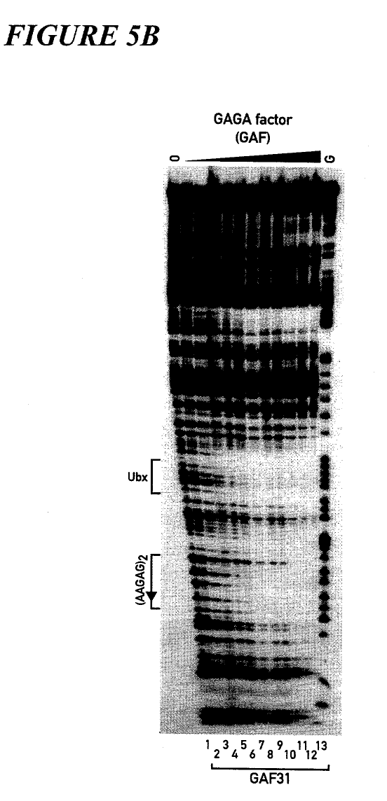

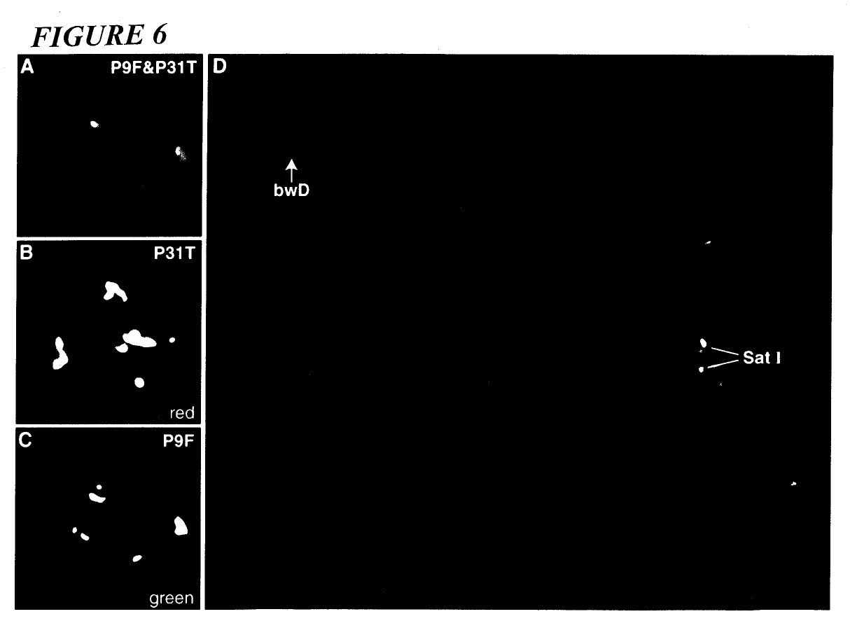

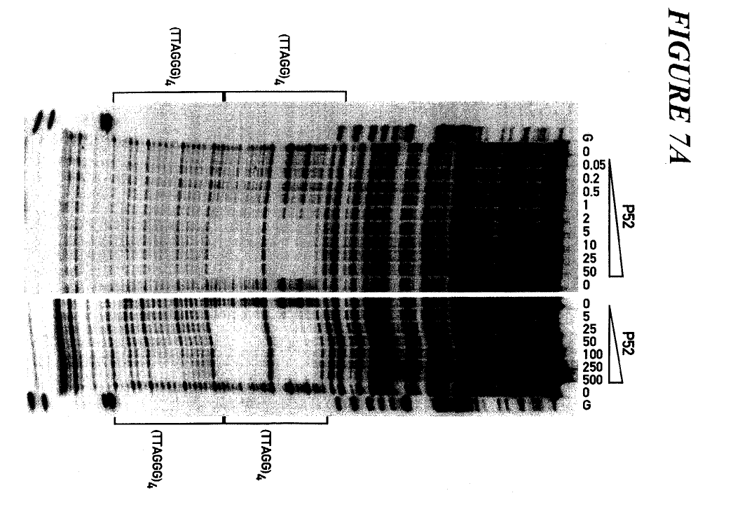

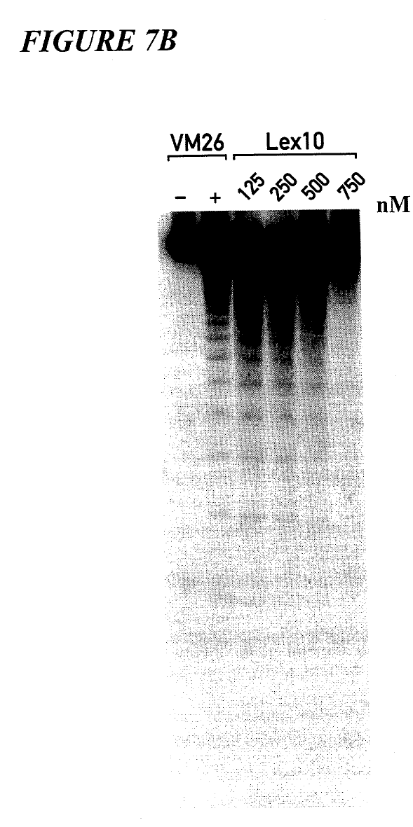

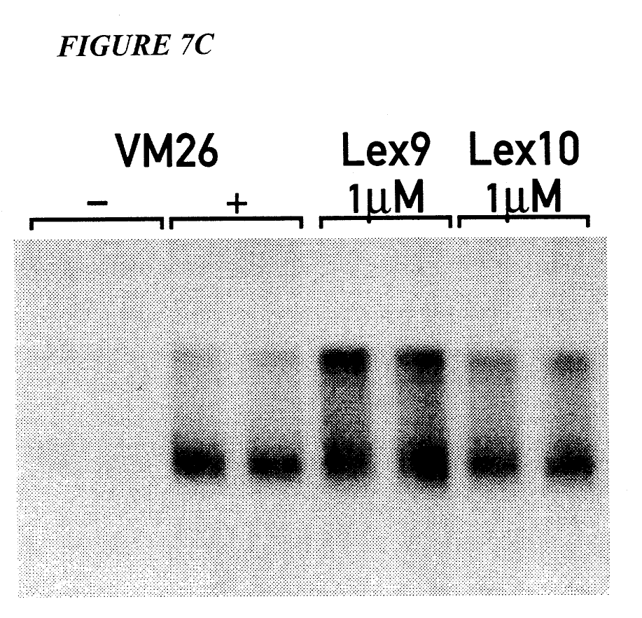

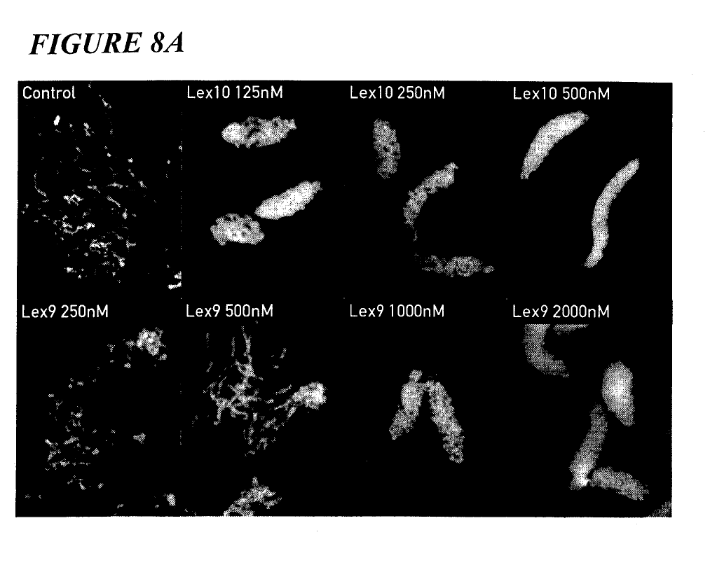

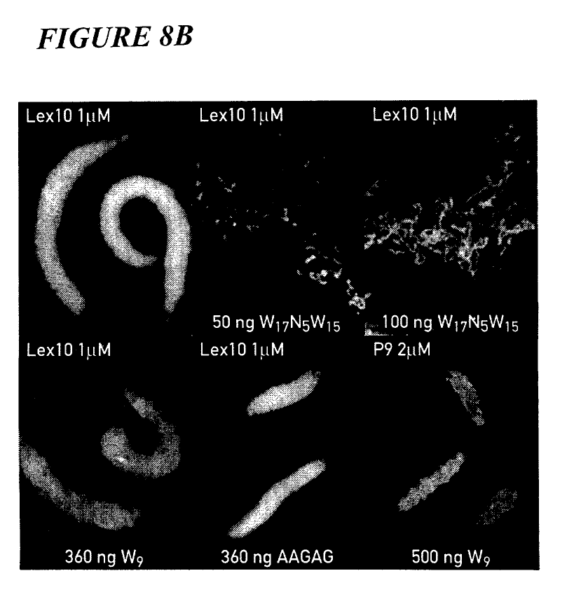

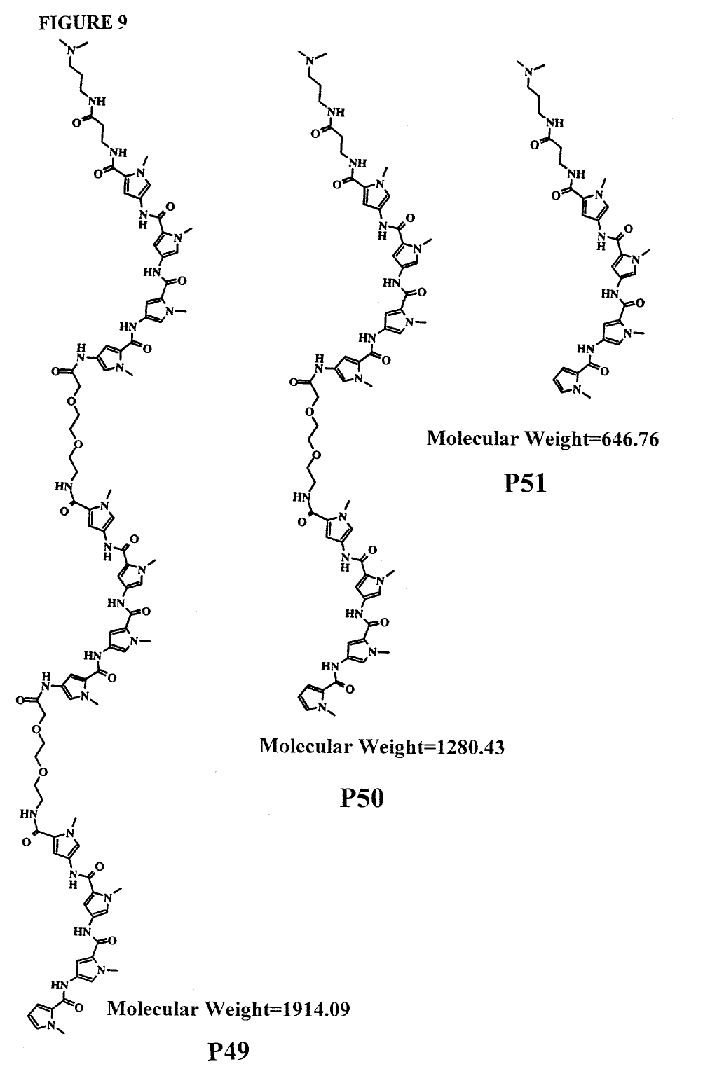

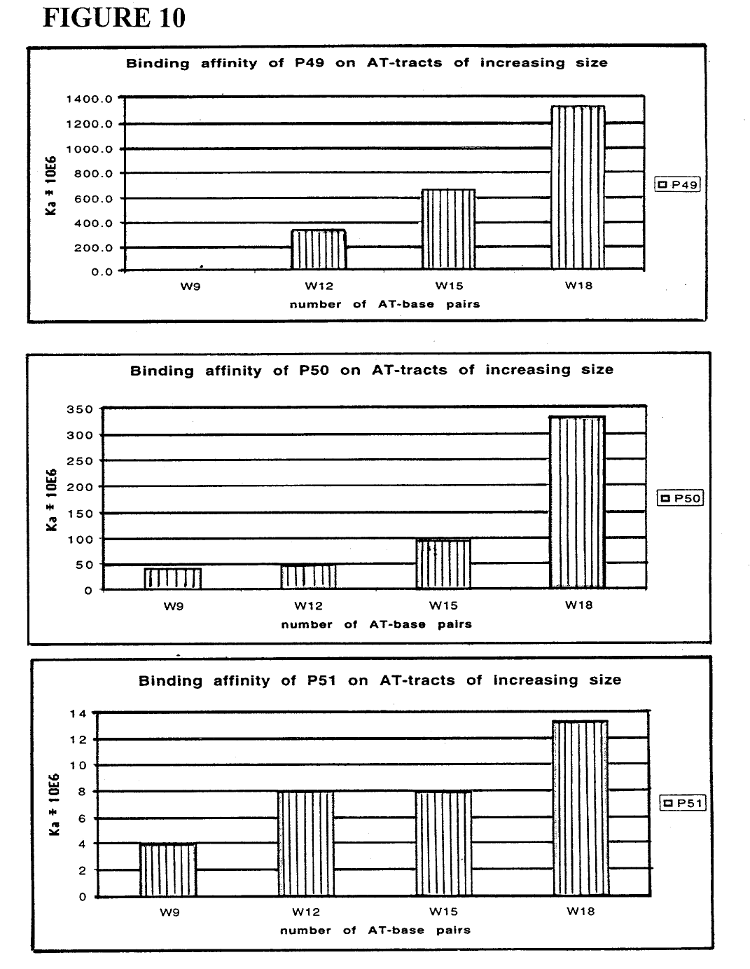

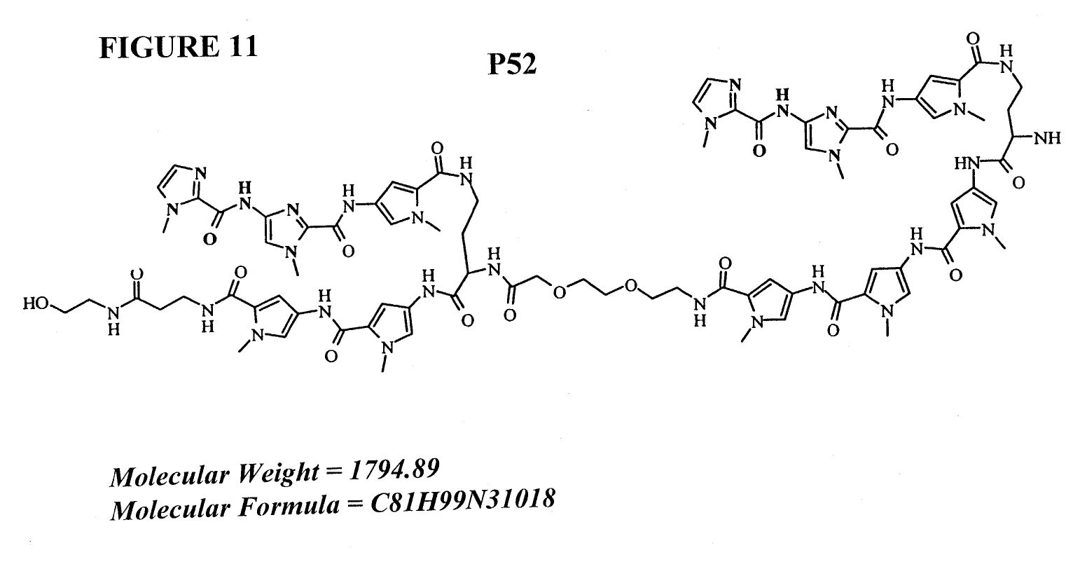

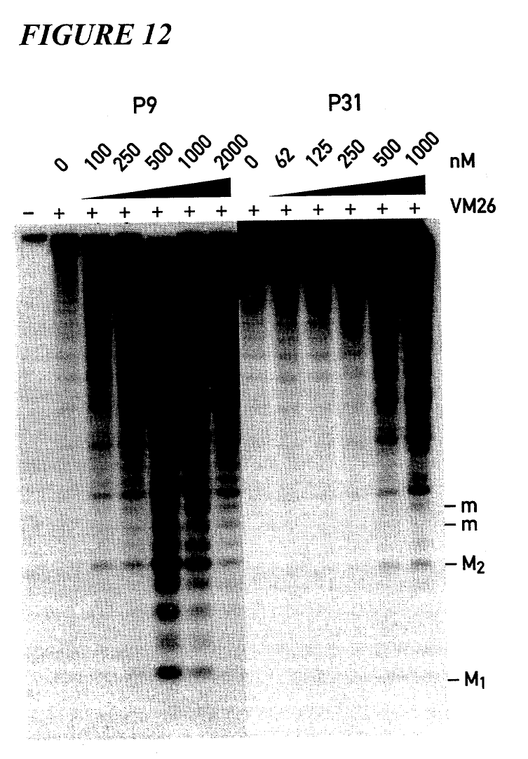

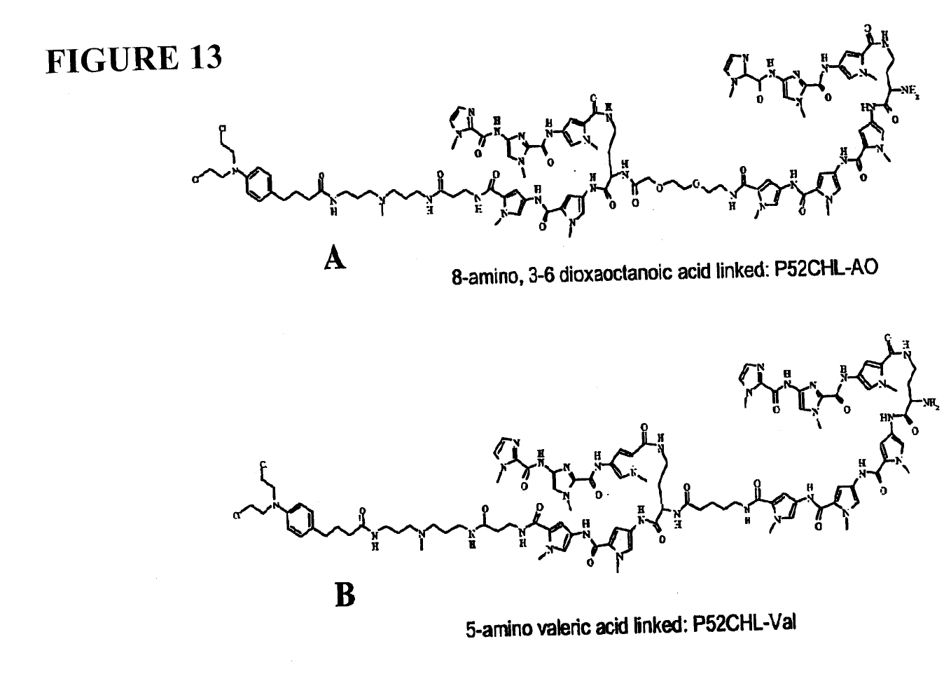

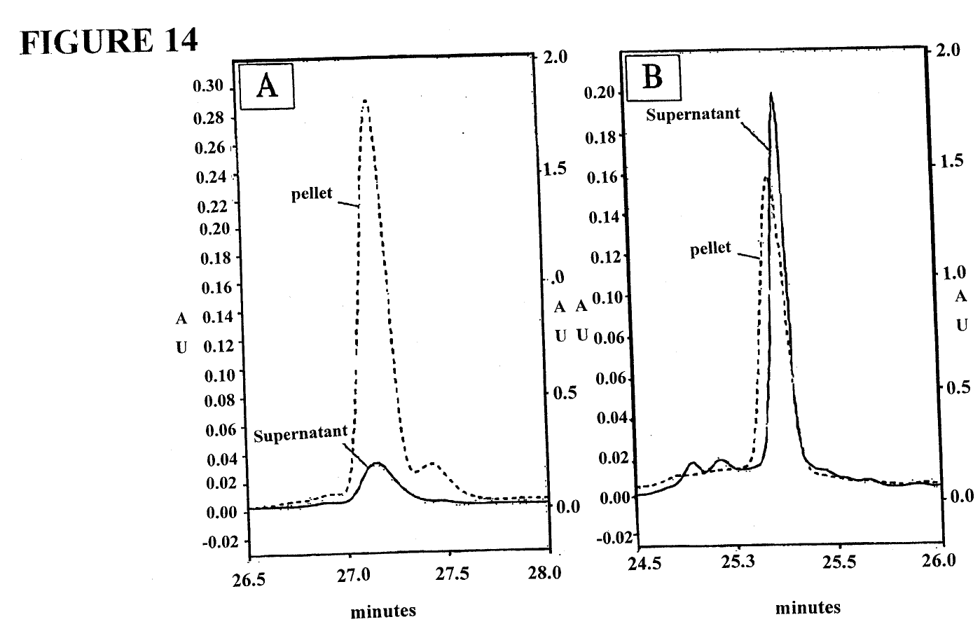

To target AT-tracts, the principal component of satellite I, III and SARs), a pyrrole pentamer was synthesized by facile solid phase chemistry in which five pyrrole (Py) aromatic amino acid rings are linked covalently by amide bonds (Baird and Dervan, 1996). The resulting compound, termed P7 had the sequence Py-Py-Py-Py-Py-β-Dp (where β=β-alanine and Dp=dimethylaminopropylamide). This compound is expected to bind 7 successive A or T base pairs (Ws) according to the n+1 rule where n is the number of amides (Youngquist and Dervan, 1985). The DNA binding properties of P7 were assessed by DNAse I footprinting experiments using a synthetic probe containing isolated, repeated AT-tracts of 9 Ws (W9, FIG. 2A). By visual inspection, the apparent dissociation constant (Kₐₚₚ) for P7 was estimated to be approximately 80 nM (Table 3).

To enlarge binding site size and improve affinity, a pyrrole hexamer termed P9 was synthesized containing a central β-alanine (PyPyPy-β-PyPyPy-β-Dp) and it was observed to bind W9 with 100-fold better affinity (Kₐₚₚ about 0.75 nM) than P7 (FIG. 2A). This latter value was obtained from footprints that extended to lower ligand concentrations than those shown in FIG. (2A).

In an attempt to further increase SAR specificity, a molecule with even more recognition units was synthesized. The resulting compound, termed P13, consisted of three pyrrole trimers linked by β-alanines (PyPyPy-β-PyPyPy-β-PyPyPy-β-Dp). P13 theoretically requires 13 Ws to accommodate all its recognition units and should therefore not bind optimally to W9. But unexpectedly, P13 binds W9 with similar affinity as compound P9 (Kₐₚₚ W9 1 nM). Furthermore, P13 displayed a marked tendency to protect GC base pairs. This unusual high GC-tolerance is evident from its footprint on the W9 probe where protection by P13 rapidly expanded from W9 into the flanking mixed sequences (FIG. 2B). This expanded protection is already noticeable at concentrations only two fold above its Kₐₚₚ (FIG. 2B). Quite striking is also the nearly complete protection (coating) of the W9 probe at higher ligand concentrations (62.5 nM and above, FIG. 2B). The non-specific behavior of P13 upon binding to short AT-tracts led the inventors to consider alternative molecular designs to target long/clustered AT-tracts.

### Example 2.

Oligopyrrole dimers exhibit significant SAR specificity

Since satellite I is composed of exclusively A and T bases, it constitutes an ‘ideal’ binding substrate for oligopyrroles. But to obtain SAR-specific compounds, molecules are required that preferably bind its clustered, irregularly spaced AT-tracts. Binding studies were carried out with P9, P7 and P13 on the Drosophila histone SAR probe which contains the following clustered/long AT-tracts (W15N3W17N5W16N13W8NW6 where N is any base, see also FIG. 3D). These studies revealed that P9, P7 and P13 had similar binding constants for the AT-tracts of the SAR probe as for W9. The ratio of these two affinities is used (KappW9/KappSAR) as an empirical measure of SAR specificity. For all compounds tested thus far, this value (referred to as SAR preference factor) was around unity (Table 3). The lack of improved affinity and specificity of P13 suggests that the phasing and or curvature correction by the two central β-alanines separating the pyrrole trimers is not optimal.

In order to target SARs more specifically with pyrrole-based drugs, alternative drag designs were explored, taking advantage of the hallmark of SARs, clustered/long AT-tracts. Compounds recognizing up to fifteen Ws (continuous or over two clustered AT-tracts) have the potential to target SARs well, since AT-tracts of 15 Ws are rare in random sequence DNA, occurring statistically only once every 33 kb for a genome with a 50% AT base composition. In SARs however, such long AT-tracts are often found. The 346-bp Drosophila histone SAR probe, for example, used in this study contains 4 AT-tracts of 15 or more Ws.

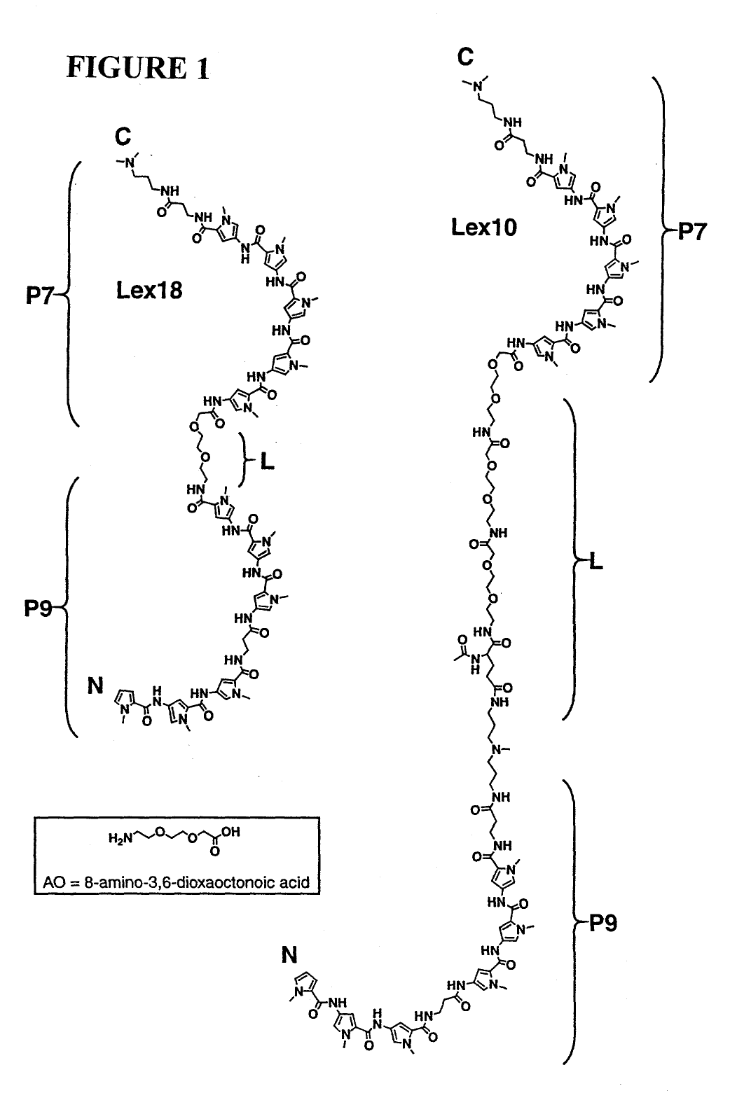

To target clustered/long AT-tracts, different means of tethering oligopyrroles into dimers with a flexible linker were tested. A suitable linker might allow bidentate binding where both covalently linked DNA binding domains (hooks) are either accommodated by a long AT-tract or interact with two clustered tracts separated by only a few nucleotides of mixed sequence. In the latter case, the linker would serve to reach across the mixed sequence spacer. A variety of possibilities were explored to synthesize oligopyrrole dimers. Satisfactory results were obtained by building up a hydrophilic, flexible linker consisting of three 8-amino-3,6-dioxaoctanoic acid units, termed AO here (FIG. 1). Molecular modelling suggested a total linker length of 60 Å in a fully extended conformation. Two oligopyrrole dimers were prepared: one by coupling P7 into a homodimer called Lex9 (PyPyPyPyPy-β-Dp-E-AoAoAo-PyPyPyPyPy-β-Dp where E=glutamic acid) and one by linking P7 and P9 into a heterodimer called Lex10 (PyPyPy-β-PyPyPy-β-Dp-E-AoAoAo-PyPyPyPyPy-β-Dp). The structure of Lex10 is shown in FIG. (1). Lex9 and 10 are expected to bind 14 and 16 Ws, respectively. As discussed, such a binding site could either be a long, continuous AT-tract or possibly be bipartite, consisting of two clustered AT-tracts. These alternative sites are referred to inclusively with the term long/clustered AT-tracts.

The relative binding affinities of dimers Lex9 and 10 for clustered/long or short/isolated AT-tracts (W9 probe) were compared by DNase I footprinting. The results are listed in Table (3). Several remarkable conclusions can be drawn from these footprinting data. While Lex10 protected the SAR-regions at subnanomolar concentration, (Kₐₚₚ0.28 nM, FIG. 3A, Table 3), a much higher ligand concentration was required to titrate the isolated W9 tract (Kₐₚₚ 20 nM, FIG. 2C, Table 3). Thus, the SAR preference factor (KₐₚₚW9/KₐₚₚSAR) of Lex10 is around 70. Note that Lex10 also discriminates against binding to the W8 tract on the SAR probe, since this site is also poorly protected (FIG. 3A).

In contrast to Lex10, For Lex9 a SAR-preference factor of only 2 was measured (Table 3). Hence, the additional pyrrole and β-alanine units that distinguish Lex10 from Lex9 must confer both improved SAR-specificity and affinity.

To examine the effect of linker length, a third heterodimer termed Lex18, was prepared by total solid phase synthesis. This compound contains the same two oligopyrrole domains (hooks) as Lex10 but is linked by only one AO unit (FIG. 1). Interestingly, although Lex18 bound the SAR region less well (Kₐₚₚ 1 nM) than Lex10, it discriminated better against binding to W9, since an improved SAR-specificity factor (Kₐₚₚ W9/Kₐₚₚ SAR) of 100 was measured for this compound (Table 3).

Importantly all dimers, in stark contrast to P13, displayed high AT-specificity and little GC tolerance. This is evident from their footprint patterns on the W9 probe. As mentioned above, P13, upon protection of W9, rapidly expanded at increasing ligand concentration into the flanking mixed sequences to eventually coat most of the probe (FIG. 2B). In contrast, Lex9 and 10 (also Lex18, not shown) hardly expand from W9 into the flanking mixed sequences and no coating is observed even at concentrations above those shown (FIG. 2C).

In summary, dimers Lex10 and Lex18, as opposed to the monomers (P9, P7 and P13) are highly SAR- and AT-specific. SAR specificity is not achieved by a significant increase in affinity for these elements but primarily by a discrimination against short/isolated AT-tracts (Table 3). These dimers are also expected to bind with high affinity to satellite I (see below).

### Example 3.

Binding mode of dimers

Attachment of the DNA cleaving moiety EDTA-Fe(II) to the C-terminus of these diners allows determination of binding location, orientation and stoichiometry by analysis of the cleavage products on high resolution gels (Taylor et al., 1984). To carry out these experiments, a Fe(II)-EDTA analogue of Lex18 (PyPyPy-β-PyPyPy-Ao-PyPyPyPyPy-β-Dp-EDTA) was prepared. The affinity cleavage results for Lex18E on the SAR probe are included in FIG. (3B) together with a G reaction. Close inspection of the cleavage products reveals that cleavage sites are predominantly at the border of large AT-tracts. By way of example, the main cleavage sites in W16 (indicating the position of the C-terminus of Lex18) are centered around nucleotide 609 (below G 607). This suggests a ligand orientation as indicated by an arrowhead on the brackets, pointing towards the N-terminal side of the molecule (FIG. 3B). For W15 and W17, the distribution of cleavage products suggests an opposite dimer orientation. These results (summarized in FIG. 3D) indicate that only a single Lex18E molecule is predominantly bound at W15, W16 and W17 (1:1 drug to DNA complex). Drug orientation must depend on the size and sequence context of a particular tract. The data do not establish whether the individual hooks of the dimer can span across a mixed sequence spacer since on this SAR Probe AT-tracts are long enough to accommodate both pyrrole hooks.

These affinity cleavage data suggest that Lex18, and by inference the other dimers, bind in an extended fashion with both hooks bound as schematized in FIG. (3E). In this binding mode, both hooks energetically contribute to binding to long/clustered AT-tracts. On shorter AT-tracts (such as W9) only one hook can be accommodated properly. The second hook remains either unbound or can interact with nearby low affinity sites. Careful inspection of the footprint data on the W9 probe is consistent with this interpretation. At high concentrations of Lex9 and Lex10, some weak protection of the mixed, relatively AT-rich region labeled M0 is observed (FIG. 2C). This protection is proposed to arise from interaction of the second hook that reaches across several unprotected base pairs. These oligopyrrole dimers can bind in an extended form to bipartite binding sites and the flexible linker can bridge several base pairs.

### Example 4.

Selective staining of DNA satellites and SARs in nuclei and polytene chromosomes

Drosophila Kc nuclei:

The footprinting data presented above demonstrated that dimeric oligopyrroles possess considerable SAR- and AT-specificity when probed on naked DNA. But does this specificity also apply to DNA packaged by histones into chromatin? To address this question, the possibility of fluorescently tagging pyrrole ligands in order to stain isolated Kc nuclei and polytene chromosomes for examination by epifluorescence microscopy was explored. If sequence preference is maintained upon tagging and also extends to chromatin, it should be possible to highlight in stained nuclei the positions of the main targets of these fluorescent oligopyrroles (satellites I and III). Moreover, the enhanced SAR preference of oligopyrrole dimers versus monomers should be demonstrated.

Fluorescent groups were coupled to monomeric and dimeric oligopyrroles using commercially available succinimidyl active esters of fluorescein. DNase I footprinting of the fluorescent ligands revealed that these derivatives are differently affected upon tagging. In general, tagging resulted in reduced binding affinity but never affected AT-specificity. Interestingly, for some compounds an improved SAR specificity factor was observed (see Table 3). For fluorescein labeled Lex10 (Lex10F), only a minor reduction in affinity and slightly altered SAR specificity was observed. In contrast, binding affinity was seriously reduced (about 50 to 100 fold) for the homodimer Lex9F and the monomer P7F (Table 3). Surprisingly, conjugation of the fluorescent label to Lex9 (Lex9F) increased its SAR preference (over W9) from 2 to a factor of 25. The SAR specificity of P9F was increased about 4 fold. The fluorescent moiety of this molecule may serve to improve discrimination.

Drosophila Kc nuclei were double stained with ethidium bromide and fluorescein-tagged pyrrole compounds (FIG. 4). To allow comparison of the dimer versus monomer staining pattern, the images by fluorescence microscopy were prepared and recorded in parallel and under identical conditions. Ethidium bromide (red) stains nuclear chromatin generally but it also markedly outlines the nucleolus due to the high RNA concentration of this subnuclear domain.

The staining patterns observed with P9F and Lex9F (green) show striking features; both ligands accumulate at one or two subnuclear locations (FIGS. 4A and B) resulting in strong green foci. These foci are generally abutting the nucleolus and are proposed to arise from the expected localization at the abundant AT-rich Drosophila satellites I and III (see below). Note that while the intensity of the foci are similar with either compound, a much stronger green signal throughout the nucleoplasm is observed with the monomer P9F. In other words, the nucleoplasm stained with P9F appears green and remains red with Lex9F. Since it is difficult to asses visually the residual nucleoplasmic staining intensity of Lex9F in the color-merged display, gray scale inserts are included in these panels that confirm the much more restrictive staining pattern and low nucleoplasmic localization of Lex9F (FIG. 4C).

The more intense nucleoplasmic localization obtained with the P9F is interpreted to arise from binding to isolated/short AT-tracts that abundantly occur throughout the genome. In turn, reduced nucleoplasmic localization of Lex9F is then a consequence of its lower preference for these tracts.

Polytene chromosomes:

Polytene chromosomes were stained with these fluorescent minor groove binding drugs to determine the subchromosomal localization of the major foci observed within Kc nuclei and could possibly allow visualization of SARs. Drosophila polytene chromosomes consist of side-by-side arrays of several hundred chromatin strands. The arms of these polytenized chromosomes consist predominantly of the euchromatic, single-copy portion of the genome. They are tethered at the chromocenter, which contains the centric heterochromatin. While the euchromatic arms are polytenized about 1000 fold, the centric repeats of the chromocenter are known to be under-replicated (Miklos and Cotsell, 1990).

FIG. (4D) shows in red (ethidium bromide, EB) the euchromatic arms and the central chromocenter of a single spread polytene chromosome. The band/interband substructure of the euchromatic arms is easily observed. This banding pattern is proposed to arise from a differential degree of DNA compaction along the arms (Rykowski et al., 1988; Spierer and Spierer, 1984). Lex9F staining (green) is superimposed over the red EB signal (total DNA). The latter pattern displays conspicuously two major signals, which abut the chromocenter. They localize to the bases of chromosomes 4 and 3R corresponding to the location of satellite I as was determined by conventional in situ hybridization (Lohe et al., 1993). Satellite I is composed of short AATAT repeats and is therefore an ideal target for Lex9F. Besides the two strong signals described, other prominent Lex9F signals (FIG. 4D) were reproducibly observed. Among those signals, one is within the chromocenter (arrowhead) and may represent the AT-rich satellite III consisting of a 359-bp repeat. In mitotic chromosomes, this satellite encompasses almost half of the X heterochromatin but is highly under-replicated in polytene chromosomes. Furthermore, a major band rich in AT-tracts can be noted on the arm of chromosome 4 (arrowhead). These observations demonstrate that Lex9F selectively stains satellite I and likely also satellite III.

It is demonstrate below that it is possible to visualize by Lex9F/10F staining genomic regions along the euchromatic arms that are rich in clustered AT-tracts supposedly representing SARs. This is particularly evident when micrographs are collected without the prominent satellites signals which tend to visually suppress the more subtle variations of red and green along the euchromatic arm. FIG. (4E) shows the band/interband structure of the polytene chromosome in red and in green/yellow the impressive staining pattern of Lex9F observed as transverse stripes of variable thickness. At some locations, an entire band is highlighted, at other sites staining occurs as a thin line at band borders or at interbands regions. Of interest are also the AT-rich signals near telomeric ends of chromosome X, 2R, 2L & 3R. We noticed that due to the much more restrictive staining of Lex9F and Lex10F as compared to EB, chromosome mapping is thereby facilitated considerably.

These epifluorescent studies of stained nuclei and polytene chromosomes strongly support the notion that proper enlargement of binding site size through dimerization of pyrrole based DNA binding elements results in an impressive gain of specificity for DNA regions with clustered, long AT-tracts. This gain in specificity is largely due to a discrimination against binding to short/isolated AT-tracts. Evidently, this specificity is maintained when DNA is packaged into chromatin.

### Example 5.

Targeting the GAGAA repeat of satellite V with P31

In the framework of a search for molecular tools to study PEV, polyamide that targets the abundant satellite V composed of GAGAA repeats (Lohe et al., 1993) was synthesised. Designing molecules that would bind to this repeat motif represented a challenge since with current knowledge, targeting of sequences containing 5′-GNG-3′ or 5′-GA-3′ with drugs composed of pyrrole and imidazole is difficult. However, successful targeting to sequences containing 5′-GTG-3′ was previously achieved using an Im-β-Im motif where β-alanine replaces the function of pyrrole (Turner et al., 1998). Since β-alanine, like pyrroles, is degenerate for A.T and T.A base pairs, we designed a compound based on these observations, to recognize a sequence composed of two tandem GAGAA repeats by systematic placement of β-alanine at the N-terminal neighbor of imidazole. The binding affinity and specificity of this compound, termed P31 (=Im-β-Im-Py-β-Im-β-Im-β-Dp), were evaluated by DNAse I footprinting. For this purpose, two different probes were examined, both containing GAGAA repeats. FIG. (5A) shows that P31 binds with subnanomolar affinity to its target binding site, in this case two GAGAA repeats (lanes 2-8). The apparent binding constant of P31 for this sequence was estimated at 0.25 nM. At higher concentrations, protection of two mismatch binding sites was observed. One of these sites contains an AAGTG motif (FIG. 5A).

To determine binding orientation and stoichiometry for P31, we prepared a Fe(II)-EDTA analogue of P31, termed P31E (Im-β-Im-Py-β-Im-β-Im-β-Dp-EDTA). Affinity cleavage was carried out on the footprint probe containing two GAGAA repeats (lane 9) and revealed one major cleavage site flanking the two GAGAA repeats, thereby confirming the assumption that one P31 molecule binds two GAGA, repeats in a 1:1 drug to DNA complex.

A drawback of this binding model, as opposed to conventional 2:1 drug to DNA complexes, is that P31 is expected to bind degenerate GC and CG base pairs, albeit with different affinity. The consensus sequence can thus be defined as SWSWWSWSWW, where S stands for a G or C and W for A or T. To evaluate binding of P31 to CACAA repeats, we used a second probe that contains two of these repeats as well as five tandem GAGAA repeats. FIG. (5A) shows that P31 protects CACAA repeats with approximately five fold lower affinity thin GAGAA repeats (lanes 11-15). Furthermore, affinity cleavage reactions using P31E revealed two major cleavage sites in the GAGAA region (lane 16), showing that in this case, two P31 molecules are bound in tandem to the pentameric GAGAA repeat. Again, it is observed than this molecule binds as a 1:1 drug to DNA complex in an orientation as indicated by arrowheads (FIG. 5A). We propose that special structural features of AT-tracts and GAGAA repeats might favor 1:1 DNA to drug complexes (see Discussion).

It was observed that P31 fed to developing Drosophila melanogaster of the brown-dominant genotype interferes with the function of the GAGA factor (GAF). A footprint experiment was therefore carried out with this protein. The DNA probe (GAF31) used for this purpose contains besides the (AAGAG)2 motif (the target of P31) a typical promoter proximal GAF binding site derived from the Ubx gene (Biggin et al., 1988). This Ubx site contains the pentameric consensus sequence GAG of GAF (Omichinski et al., 1997). The DNase I footprint studies show that, while GAF binds both the (AAGAG)2 and Ubx motifs, P31 interacts only with the former satellite repeats (compare panels A and B of FIG. 5).

Selective Staining of GAGAA Satellite V in Nuclei and Polytene Chromosomes

We synthesized fluorescent derivatives of P31 to visually assess their binding targets by staining of nuclei and chromosomes. DNase I footprinting of the fluorescent ligands revealed that P31T bound the GAGAA sequence with unaltered specificity but with 100 fold reduced binding affinity. Drosophila Kc nuclei were triple stained with DAPI, Lex9F and P31T and recorded by epifluorescent microscopy. The micrographs obtained again are striking since one notes against the blue DAPI background of nuclear DNA, separate green and red foci stemming from Lex9F and P31T staining, respectively (FIG. 6A). Closer inspection reveals that these foci are largely non-overlapping (compare panels A and B).

In situ hybridization analysis showed that it is possible to detect satellite I but not satellite V ((GAGAA)n) in polytene chromosomes obtained from wild type flies, supposedly due to a more severe under-replication of satellite V (Platero et al., 1998). Hence, due to this apparent absence of GAGAA repeats, the specificity of P31T for its target binding site cannot be evaluated using ‘normal’ polytene chromosomes. Therefore, to circumvent this limitation, we prepared polytene chromosomes from bowndominant (bwD) flies which harbor an large block of heterochromatin (about 1.7 megabases) composed of GAGAA repeats inserted into the coding region of the brown (bw+) gene. This heterochromatic insert appears to be normally polytenized (Csink and Henikoff, 1996; Dernburg et al., 1996; Platero et al., 1998) probably due to its euchromatic localization, Polytene chromosomes were prepared from these flies and stained with P9F, P31T and DAPI, The results obtained were striking (FIG. 6). P31T (red) highlighted conspicuously the bwD GAGAA insert at locus 59E on the right arm of chromosome 2 (2R). No other P31T foci were observed, neither at the chromocenter nor along the euchromatic arms. Lex9F (green) marks the position of satellite I at the base of chromosome 4 and 3R, abutting the chromocenter as shown above (FIG. 6). The familiar band/interband pattern of polytene chromosomes is revealed in blue by DAPI staining.

In summary, different satellite-specific polyamides were synthesized as established by footprinting and epifluorescence microscopy. Oligopyrrole dimers (and their monomers) target mainly satellite I, III and SARs. Enhanced SAR-specificity was obtained by tethering oligopyrroles moieties with a flexible linker. The Im-Py compound P31 was shown to specifically bind satellite V. All these compounds bind their DNA targets as 1:1 drug to DNA complexes.

### Example 6.

Oligopyrroles mediate chromatin remodelling and inhibit topoisomerase II cleavage in a sequence-specific fashion

Exposure of nuclei to distamycin (Py-Py-Py) causes opening of the chromatin fiber, thereby facilitating cleavage by restriction enzymes and topoisomerase II at satellite III (Kas and Laemmli, 1992). Do synthetic polyamides have similar effects on chromatin? As mentioned above, satellite III consists of 359-bp repeats and each repeat unit is packaged in two nucleosomes. Biochemically, satellite III repeats behave as SARs; they preferentially bind nuclear scaffolds, topoisomerase II, HMG-I/Y and MATH20 (Girard et al., 1998; Kas and Laemmli, 1992). Topoisomerase II is also enriched at satellite III in vivo, as demonstrated by microinjection of fluorescent topoisomerase II into Drosophila embryos (Marshall et al., 1997). Satellite III contains one prominent topoisomerase II cleavage site per repeat located in every second nucleosomal linker (Kas and Laemmli, 1992). Topoisomerase II cleavage products accumulate in the presence of the cytostatic drug VM26 when Kc nuclei are exposed to Xenopus egg extracts, rich in topoisomerase II. This treatment generates a DNA ladder with a repeat length of 359 bp as revealed by hybridization. The ladder is observed only upon addition of VM26 (FIG. 7A, left). Interestingly, cleavage is massively stimulated by addition of the monomer P9 (also P7, not shown). Cleavage stimulation is evidenced by an increased intensity of the main repeat band (marked M, one cut per 359-bp repeat) and a shift of the ladder to shorter fragments. Stimulation is maximal at 500 nM and starts to diminish at higher concentrations (FIG. 7A). P9 exposure also results in the appearance of additional, minor bands (marked m) that most likely arise from cleavage within nucleosomes (see discussion). These minor bands are not observed without the drug, even after extended exposure (data not shown).

Next, the potency of P31 was tested in this assay. The results, shown in FIG. (7A), demonstrate that P31 stimulates cleavage considerable less well than P9. That is, while, massive cleavage stimulation is observed with the lowest concentration of P9 (62 nM, FIG. 7A, lane 3), no significant reinforcement of the pattern is observed with P31 up to a concentration of 200 nM (FIG. 7A, lanes 8 to 11). Only at 500 nM is cleavage stimulation by P31 comparable to that obtained with 62 nM of P9 (compare lane 3A to lane 12). Stimulation with P9 is maximal at 500-1000nM and starts to diminish at higher concentrations. The cleavage ladder induced by P31 at these concentrations is also less pronounced than that of P9 in keeping with the dose response observed. These dosage experiments Demonstrate that P9 opens the heterochromatic satellite III at a roughly 10 fold lower concentration than P31. FIG. (7B) shows a similar experiment with dimers Lex10. Interestingly it was observed that Lex10 does not stimulate topoisomerase II cleavage and that inhibition occurs abruptly around 600 nM (FIG. 7B).

The data presented above demonstrate that the synthetic oligopyrrole compounds P9 and P7 (not shown) strongly facilitate cleavage by topoisomerase II. The dual response (stimulation or inhibition of enzyme activity) to drug treatment is thought to reflect the initial opening of chromatin, facilitating cleavage, whereas inhibition of cleavage at higher concentration is proposed to arise from blocking of the actual cleavage sequence by these minor groove binding drugs. An important control experiment was carried out to rule out that cleavage stimulation by P9 occurs through chromatin opening and not by effecting directly the overall enzymatic activity of topoisomerase II. Double-stranded topoisomerase II cleavage during exposure of cells or nuclei to VM26 mediates the accumulation of genomic fragments that can be observed by the appearance of a 50 to 100 kb DNA smear using pulse-field electrophoresis. If inhibition of topoisomerase II cleavage by Lex10 is specific for SARs such as satellite III, than the intensity of the smear caused by genome fragmentation should not be affected. FIG. (7C) further shows in duplicate the appearance of the 50 to 100 kb DNA smear following addition of VM26 (lanes 3 and 4). This band is absent when VM26 was omitted (lanes 1 and 2). We observed that the 50 to 100 kb DNA smear in the presence of Lex10 (lanes 7 and 8) and also Lex9 (lanes 5 and 6) was not visually altered. Thus, although Lex10 at the concentration used (1 μM) inhibits cleavage of topoisomerase II completely in satellite III (FIG. 7B), it does not interfere with the genome-wide cleavage.

An additional observation that supports the notion of chromatin opening is that P9 also facilitated cleavage within satellite III by restriction enzymes. Satellite III repeats contain near the topoisomerase II cleavage site a HaeIII restriction sequence. It was previously been demonstrated that cutting by HaeIII in chromatin (not DNA) is facilitated by distamycin (Kas and Laemmli, 1992). We made a similar observation using P9 (data not shown).

### Example 7.

Specific inhibition if chromosome condensation

Mitotic Xenopus egg extracts convert added nuclei and sperm to chromatids in vitro. This chromosome condensation process requires topoisomerase II (Adachi et al., 1991), the protein complex condensin (Hirano T, 1997) and presumably other unidentified activities present in the mitotic extract. First, chromatin is remodeled and nuclei then proceed quite synchronously through a number of morphologically distinguishable steps (Hirano and Mitchison, 1991). Remodeling is morphological manifested by swelling of the nuclei which involves exchange of basic sperm-specific proteins for H2A/H2B and the incorporation of histone B4 (Dimitrov S, 1994).

Pyrrole drugs were added to the extract together with the sperm or after the remodeling step (at 10 minutes) and the extent of condensation was determined after 120 min. At this time point, the conversion of all sperm nuclei to clusters of individual chromatids is complete in the absence of drug (FIG. 8A, control). Lex10 was found to be a potent inhibitor of chromosome condensation. Addition of this compound at 125 to 250 nM (indicated) arrested this process at the so-called early ‘ruffle’ stage (FIG. 8A). These structures retain the swollen sperm shape, but they have peripheral blebs (ruffles) and a slightly heterogeneous interior. At this drug concentration, no chromatids are seen. If the concentration of Lex10 is raised to 500 nM, we observed an even earlier arrest as evidenced by the accumulation of swollen, remodeled sperm-shaped nuclei containing a homogeneous interior and smooth periphery. Lex9, less SAR specific than Lex10 according to the footprinting data, was found to be a less potent inhibitor of condensation since it requires 4 to 8 fold higher concentration (I to 2 μM) to achieve a block at the ruffle stage (FIG. 8A). Little inhibition was observed with Lex9 at a lower dose of 250 to 500 nM. The monomer P7 was also tested but we observed no inhibition with the pyrrole pentamer up to the highest concentration (8 μM) tested. Condensation was inhibited at a ruffle stage with a P9 concentration of 2 μM (not shown).

Is inhibition of condensation by pyrrole compounds a specific process? The fact that the concentration of a given drug, required for complete arrest of condensation is related to the SAR preference factors suggest that the inhibition is specific. To address the question of specificity directly, competition experiments were performed. Preliminary competition assays showed that chromosome assembly in egg extracts is relatively insensitive to added oligonucleotides (about 50 bp in length). Up to 500 ng of oligonucleotides can be added to the extract containing sperm nuclei (about 75 ng DNA) without interfering with condensation. We therefore argued that, if inhibition by oligopyrroles occurs through binding to clustered AT-tracts, addition of an oligonucleotide containing clustered (not single) AT-tracts should prevent the arrest.

In the experiment shown in FIG. 8B, Lex10 was added to the extract at a final concentration of 1 μM (several fold above the minimum inhibitory concentration) after which competitor oligonucleotides were added at different stages. Three different oligonucleotides of similar size were used: the SAR oligo contains two large clustered AT-tracts of the SAR probe (W17N5W15), the W9 oligo has a single AT tract of 9 base pairs and the GAGAA oligo harbors 5 tandem GAGAA repeats.

FIG. (8B) shows that condensation inhibition by Lex10 is completely reversed by the addition of 50 to 100 ng of the SAR oligo whereas up to 9 times this amount (360 ng) of either the W9 or GAGAA oligo did not reverse the block. This supports the assumption that Lex10 interferes with chromosome dynamcis by selective titration of long, clustered (not isolated) AT-tracts and that inhibition does not occur through general DNA binding. This contrast with the observation made with the monomer P9, which blocked condensation at 2 μM. Addition of 500 ng of either of the SAR-, W9- or GAGAA-oligo did not rescue chromatid assembly. Hence, P9 interferes with condensation in a sequence independent manner.

Biochemical analysis of the arrested sperm-derived structure demonstrated that it contained a normal protein composition concerning topoisomerase II and the components of condensin (not shown).

In conclusion, the data demonstrate that the dimer Lex10 specifically interferes with chromosome condensation through interaction with clustered, long AT-tracts. It further highlights the experimental potential of pyrrole-imidazole based drugs as powerful tools for chromosome research and cell biology.

### Example 8.

Tandem-linked linear molecules

The use of a longer 8-amino-3,6-dioxaoctonoic acid linker (referred to as Ao), bridging 2-3 base pairs per Ao unit, proved to be excellent way of joining DNA binding elements without impairing sequence preference of the individual units. For this binding study, three compounds were synthesized with one, two and three DNA binding elements (an N-methylpyrrole carboxamide tetramer) that were covalently linked by longer amphipathic linker mentioned above (Ao). These pyrrole-based compound are degenerate for A and T. The trimeric compound P49 (see FIG. 1) showed very little preference for these sequences. The dimeric compound P50 display intermediate properties. This is illustrated in FIG. 10. The methods of synthesis are the same as those described in Examples 1 to 7.

### Example 9

Tandem linked hairpin molecules

A hairpin shaped molecule designed to target 5′-GGTTA-3′ will have only moderate binding affinity and sequence specificity. Targeting a longer sequence such as 5′-GGTTAGGTTA-3 with two tandem linked hairpins (the DNA binding element) greatly increases binding affinity and sequence specificity. As above, optimal results were obtained by use of a Ao linker; The structure of this tandem hairpin molecule (termed P52) is shown in FIG. 11. The excellent sequence specificity of P52 for 5′-GGTTAGGTTA-3 is shown in a DNAse I footprinting experiment in FIG. 12. In this Figure, it can be observes that at concentrations far above the concentration required for protection (˜5 nM), no additional site become protected, even at highest concentration tested (500 nM). The methods of synthesis are the same as those described in Examples 1 to 7

Using this approach, the relative low sequence specificity of Pyrrole-Imidazole compounds can be overcome and compounds with enough affinity and specificity for biological applications can be obtained.

### Example 10

Quantification of enhanced solubility conferred by the amphipathic linker

An important property for linked polyamides is adequate solubility in aqueous solution, such as tissue culture media. Tethering polyamides with an amphipathic linker of the invention, in contrast to a hydrophobic linker, can confer enhanced solubility to the DNA-binding molecule.

By way of example, two tandem hairpin polyamides (“P52” as described in Example 9), recognizing two insect-type telomere repeats (TTAGGTTAGG) were synthesized and equipped with a hydrophobic, alkylating group (Chlorambucil) as “effector moiety”. One compound contains a hydrophobic methylene linker (5-amino valeric acid) and the other an amphipathic linker of the invention (8 amino-3,6-dioxaoctonoic acid or “AO” for short). The structures are shown in FIG. 13.

In tissue culture experiments, designed to measure the cytotoxicity of the above compounds, it was observed that P52CHL-Val, in contrast to P52CHL-AO, precipitated; this is manifested by the formation of crystals adhering to cells and to the bottom of the culture dish.

To quantify the enhanced solubility, both compounds were dissolved in cell culture medium, supplemented with serum (RMPI medium with 5% NCS, 200 μL final volume) at a concentration of 5 μM (by dilution from a 1 mM stock in DMSO). After an incubation period of 4 hours at 25° C., the solutions were spun at 4° C. (16'000 g, 5 min) and the supernatants transferred to new tubes. The insoluble pellet was taken up with 100 μL acetonitril (90% in water). The fraction of precipitated compound (in the pellet) and soluble compound (in the supernatant) were determined by HPLC integration. The results are plotted in Table 4 below and FIG. 14. The results demonstrate that solubility is approximately 5 fold higher for the compound with the amphipathic linker of the invention.

TABLE 4
Soluble in insoluble fraction of differently
linked tandem hairpin polyamides.

| | Percentage in | Percentage in |
|---|---|---|
| Linker | pellet | supernatant |
| B amino-3,6- | 46 | 54 |
| dioxaoctonoic acid | | |
| 5-amino valeric acid | 89 | 11 |

### Example 11

Materials and Methods

The following indicates the materials and methods used throughout the Examples.

Boc-β-PAM-resin, HBTU, Fmoc-Glu(otBu)—OH, Boc-β-alanine and Boc-γ-aminobutyric acid were purchased from Novabiochem AG, Switzerland. HOBt was from Bachem. The methylester of 4-amino-1-methylpyrrole-2-carboxylic acid hydrochloride was synthesized by Bachem on special request. DMF, acetonitrile (HPLC grade) and 3,3′-diamino-N-methyldipropylamine were purchased from Aldrich. N,N-diisopropylethylamine (DIEA) was from Sigma and Fmoc-8-amino-3,6-dioxaoctonoic acid was purchased from Neosystem, France. Dichloromethane (DCM), thiophenol (PhSH), ethanedithiol (EDT), trifluoroacetic acid (TFA), thiodiglycol, piperidine, N,N′-diisipropylcardodiimide (DIC), dicyclohexylcarbodiimide (DCC) and 3-dimethylamino-l-propylamine were from Fluka. FLUOS (5(6)-carboxy-fluorescein-N-hydroxysuccinimide ester) was purchased from Boehringer-Mannheim. All reagents were used without further purification. Glass peptide synthesis reaction vessels (5 ml) with a #2 sintered glass filter frit were obtained from Verrerie Carouge (Geneva, Switzerland). Analytical and semi-preparatory HPLC was performed as previously described (Baird and Dervan, 1996). Electrospray Ionization mass spectra were obtained in the positive ion mode on a Trio 2000 instrument at the University Medical Center (Geneva, Switzerland).

Syntheses of pyrrole monomer for solid phase synthesis.

1,2,3-Benzotriazole-1-yl 4-[tert-Butoxycarbonyl)amino]-1-methylpyrrole-2-carboxylate or Boc-Py-Obt was synthesized from 4-amino-1-methylpyrrole-2-carboxylic acid methylester hydrochloride (Baird and Dervan, 1996).

Manual Solid phase synthesis of pyrrole compounds

Couplings of Boc-Pyrrole were performed as previously described (Baird and Dervan, 1996). Boc deprotections were carried out with 90% TFA, 5% EDT and 5% PhSH (2×30 s, 1×20 min). All Fmoc amino acids constituting the linker part were coupled after pre-activation with 1.1 equivalents of HOBt and DIC for 5 min. The obtained in situ active esters were added to the deprotected and neutralized resin in 4 fold excess and allowed to react for 1×1 h and 1×30 min in the presence of 8 equivalents DIEA. The temporary Fmoc protecting group was removed with 40% piperidine in DCM (1×60 s, 1×10 min). The resin was then washed with DCM (3×) and DMF (3×). The N-amino group of glutamic acid was acetylated (2×15 min) with acetic anhydride (2:2:1 DMF/Ac2O/DIEA). The t-butyl protecting group of glutamic acid was removed as described above for Boc groups. Cleavage from the resin with 3-dimethylamino-l-propylamine or 3,3′-diamino-N-methyldipropylamine was performed as described (Baird and Dervan, 1996). After cleavage, most of the excess organic base was removed prior to HPLC purification by precipitation of pyrrolic peptides. For this purpose, the reaction mixture was mixed with 3-4 volumes of DCM, followed by the addition of 10 volumes of cold (−20° C.) petroleum ether. The precipitated product was collected by centrifugation and dissolved in 1% TFA to obtain acidic pH.

Dimerization of oligopeptides.

First, all purified oligopeptides (with a unique reactive carboxyl or amine) were loaded an additional time on a preparative HPLC column and washed extensively with 20-30 column volumes of TFA-free buffer A (5 mM HCl in water) to eliminate traces of remaining cleavage reagent and TFA that would otherwise terminate the dimerization reaction. The compounds were eluted with buffer B (2 mM HCl, 90% acetonitrile), collected, lyophilized and dissolved in DMF at a concentration of 20-50 mM. The concentrations of pyrrole pentamers were determined spectrophotometrically assuming an extinction coefficient of 46000 M-1 at 312 nm (Martello et al., 1989). Concentrations of compounds containing (Py)3-βR-(Py)3 were determined spectrophotometrically assuming an extinction coefficient of 68000 M-1 at 302 nm. For activation of the oligopeptide containing the unique carboxyl (N-terminal glutamic acid), 300 to 500 nmoles were mixed with 4 equivalents of HOBt (1M in DMF) and 4 equivalents of DIC (3M in DMF) and incubated at room temperature for 15 min. Next, DIEA was added to obtain an apparant pH of approximately 10 (between 0.4 and 0.8 μl) and the oligopeptide containing the unique primary amine was added (same equimolar amount as other oligopeptide). The mixture was incubated at 37° C. in a shaker at 1000 RPM. Aliquots were taken (−0.1 μl) to follow the formation of dimer by RP-HPLC). The reaction time for >95% completion varied between several hours and o/n. When the reaction was complete, the dimeric oligopeptide was purified (by RP-HPLC) and dried in vacuo. Dimeric oligopeptides were dissolved in DMF containing 0.1% (v/v) thiodiglycol at a concentration of 1.00 mM and stored at −70° C. The extinction coefficient of the oligopeptide dimer was taken as the sum of the two extinction coefficients of the oligopeptide monomers. The recovery was usually between 25 and 50%. All dimers were analysed by ESI-MS.

Fluorescein-labeling of compounds.

Oligopyrroles with a unique primary amine were obtained by either cleavage of oligopeptides from solid phase with a diamine (3,3′-diamino-N-methyldipropylamine) or deprotection of an N-terninal γ-aminobutyric acid spacer. The N-hydroxy succinimide active ester of fluorescein was added in 3 fold excess together with 6 or more equivalents of DIEA. Reactions were allowed to proceed at room temperature for 15 minutes and the fluorescein labeled oligopeptide was purified by HPLC.

Synthesis of P31 and P31T

P31 (Im-β-Im-Py-β-Im-β-Im-β-Dp) was synthesized in a stepwise fashion by manual solid-phase synthesis from Boc-β-PAM resin as previously described for Imidazole and Pyrrole containing hairpin polyamides (Baird and Dervan, 1996). Since acylation of the imidazole amine on solid phase gives unsatisfactory results, Boc-β-alanine couplings were performed by preparing a Boc-β-Im—OH dimer in solution. The synthesis and activation was as described for dimers of Boc-γ-aminobutyric acid and Imidazole (Baird and Dervan, 1996). For fluorescent labeling of P31, cleavage from the solid support was performed with 3,3′-diamino-N-methyldipropylamine. After HPLC purification, the C-terminal amine was acylated using an commercially available (Molecular Probes) N-hydroxy succinimide active ester of Texas red. The resulting compound was then again purified by HPLC.

Preparation of probes for DNase I footprinting.

Synthetic oligonucleotides:

| | |
|---|---|
| | GATCTAGACGCATATTAATTGCGCTGTCGACGCATTAGTG |
| and: | |
| | GATCCACTAATGCGTCGACAGCGCAATTAATATGCGTCTA |

were hybridized to obtain the W9 probe, oligomerized by ligation and digested with BamHl and BglII to obtain different tandem repeats. The following oligonucleotides were prepared identically:

GAF31 is composed of the oligonucleotides:

```
                GATCCTCAGAGAGAGCGCAAGAGCGTCCCGGGAGAAGAGAAGAGAGTA
and
                GATCTACTCTCTTCTCTTCTCCCGGGACGCTCTTGCGCTCTCTCTGAG
and BrownI of o1igonucleotides:
              GATCCAAGAGAAGAGAAGAGAAGAGAAGAGTACTTATTAACACAACACA
and
            GATCTTGTGTTGTGTTAATAAGTACTCTTCTCTTCTCTTCTCTTCTCTTG.
```

Fragments were purified on low-melt agarose gels and then cloned into a modified pSP64 vector, cut by BamHI and BglII. End-labeling was carried out following digestion with HindIII and a fill-in reaction with Klenow DNA polymerase. The labeled plasmid was cut with PvuII and the target fragments purified from low-melting agarose gels. The 657 bp EcoRl/Hinf1 fragment of the Drosophila histone SAR was cloned into the SmaI site of the modified pSP64 plasmid. This SAR probe was end-labeled following digestion with EcoRl, then cut with ClaI and the resulting 347 bp fragment purified from low-melting agarose gels.

DNase I footprinting.

All reactions were performed in a total volume of 40 μl. A polyamide stock solution or buffer (for reference lanes) was added to an assay buffer containing 20 kcpm radiolabeled DNA, affording final concentrations of 10 mM Tris-HCl (pH 7.4), 10 mM KCl, 10 mM MgCl, 5 mM CaCl2, 0.5 mM EDTA, 0.5 mM EGTA, 1 mM DTT and 0.1% digitonine. The solutions were allowed to equilibrate for at least 2 h at room temperature. Footprinting reactions were initiated by the addition of 2 μl of a DNase stock solution (containing −100 pg DNase I in buffer) and allowed to proceed for 2 min at room temperature. The reactions were stopped by addition of 10 μl of a solution containing 1.25 M NaCl, 100 mM EDTA. Next, 5 μl of a 1% SDS solution was added, followed by 2 μl of a solution containing 1 μg poly(dA-dT), 1 μg salmon sperm DNA and 10 μg glycogen and the DNA was ethanol precipitated (20 min at −20° C.). The reactions were resuspended in 4 μl of 80% formamide loading buffer, denatured 10 min at 85° C., cooled on ice and electrophoresed on 8% polyacrylamide denaturing gels (5% cross-link, 8 M urea) at 30 W for 1 h. The gels were dried and exposed o/n at −70° C.

Staining of Drosophila nuclei and polytene chromosomes.

Kc Drosophila nuclei were isolated (Mirkovitch et al., 1984), diluted into XBE (10 mM Hepes, pH 7.7, 2 mM MgCl2, 0.1 mM CaCl2, 100 mM KCl, 5 mM EGTA and 50 mM sucrose), fixed with 0.8% fresh paraformaldehyde for 15 minutes and spun onto a round coverslip (10 mm) as described previously (Boy de la Tour and Laemmli, 1988). For washing and staining, coverslips were floated on 60 μl drops of XBE deposited on parafilms. After centrifugation coverslips were washed twice (1 minute), stained for 60 minutes, washed four times (1 minute) and then mounted in PPDI (5 mM Hepes pH 7.8, 100 mM NaCl, 20 MM KCl; 1 mM EGTA, 10 mM Mg SO4, 2 mM CaCl2, 78% glycerol, 1 mgr/ml paraphenylene diamine). FIG. (4) panel A was stained with 0.5 μM P9F and 15 μM ethidium bromide (EB). Panel B was stained with 1 μM Lex9F and 15 μM EB.

Squashed polytene chromosomes were prepared from late third instar larvae salivary glands and stained with fluorescent oligopyrroles as follows. Chromosomes were rehydrated by overlayering 60 μl of XBE for 15 minutes. To avoid drying, a cover slip was applied which was wedged up with two other cover slips positioned on either side of the squash area. Staining was carried out identically during 60 minutes in 60 μl XBE using various concentrations Lex9F, ethidium bromide and/or DAPI. This solution also contained 30 μg/ml of RNase A to avoid RNA signals. Slides were washed twice (7 minutes) in 50 ml of XBE and mounted with PPDI. The following final dye concentration were used: FIG. (4), panel D Lex9F 16 μM and EB 30 μM, panel E Lex9F 1 μM and EB 30 μM. Images were recorded with a wide field, deconvolution-type imaging system from DeltaVision.

Other Methods

Topoisomerase II inhibition and chromosome assembly were as described previously (Girard et al., 1998, Strick and Laemmli, 1995). Affinity cleavage experiments was performed as described elsewhere (Turner et al., 1997).

### DISCUSSION

The potential of sequence-specific minor groove binding polyamides as novel tools to address issues of chromosomal structure, dynamics and the biological functions of non-genic DNA was explored. To this end, compounds that interact with satellite I (AATAT), V (GAGAA) and SARs, including the SAR-like satellite III were synthesized. Although targeting satellite I and SARs can be achieved with ‘conventional’ minor groove binding drugs such as Distamycin, Hoechst and DAPI, their relatively short binding site give rise to high background signals. Increased binding site size was shown to confer high specificity for long AT-tracts as found is these satellites and SARs. Impressive targeting to SARs was achieved by linking two oligopyrroles moieties with a flexible linker to form dimers. Lex10 and 18, contain identical DNA-binding elements; a pyrrole pentamer (P7) and a pyrrole hexamer (P9), but differ only in their spacer length (FIG. 1). Both diners bound SARs nearly two orders of magnitude better than W9 (Table 3). No significant SAR-specificity was obtained with monomeric oligopyrroles but this is expected since they fit equally well to W9 as to the longer AT-tracts of SARs. The data suggest that oligopyrrole dimers bind SARs in an extended bidentate binding mode where both hooks are either accommodated by a single/long or by two clustered AT-tracts (bipartite binding site, FIG. 3e). SAR-specificity is then due to an energetically favorable interaction with both hooks in bipartite/long and a less favorable interaction at short/isolated AT-tracts, where only one hook is bound. The footprint studies with dimers are in line with a monodentate binding mode at W9, since at high ligand concentration, the protection in the flanking region (M0) is proposed to arise from the ‘free’ hook (FIG. 2C). Studies to dissect the binding mode of these dimers in more detail confirm the extended binding mode and demonstrate that the flexible linker can bind bipartite binding sites separated by several base pairs.

Importantly, Lex10 and 18 displayed high AT-specificity and low CC-tolerance. This observation contrasts with that of monomer P13 which consists of three pyrrole trimers linked with β-alanines. P13 was found to be very GC-tolerant since its footprint expanded rapidly at increasing ligand concentration from W9 into the flanking mixed sequences to eventually protect (coating) the entire probe (FIG. 2B). This molecule requires theoretically an AT-tract of 13 Ws. It is proposed that about 9 minor groove recognition units fit well into W9 and that this relatively favorable interaction then ‘force feeds’ the remainder of the molecule along the minor groove. In contrast, the long flexible spacer of the oligopyrrole dimers may provide the molecular freedom to avoid continuation in the minor groove. Several publications previously described the joining of netropsin and distamycin to dimers with different linkers to achieve binding to sites of 8 to 10 Ws (Neamati et al., 1998; Wang and Lown, 1992). The experiments presented here demonstrate that flexible, ethylene oxide-type spacers of the oligopyrrole dimers are highly suited to target continuous or bipartite AT-tracts of 15 to 18 Ws with good specificity.

Synthesizing compounds that bind GAGAA repeats with high affinity is chemically more challenging since this sequence includes a ‘difficult’ motif. However, impressive targeting to satellite V repeats was obtained with the monomer P31 which is composed of both imidazole and pyrrole units (FIG. 1B). Structurally, P31 extends recent observations that the ‘difficult’ triplet GWG sequence can be targeted by a Im-β-Im motif where β-alanine is positioned N-terminal of imidazoles (Turner et al., 1998). In P31, this design principal was systematically extended to achieve subnanomolar affinity for two consecutive GAGAA repeats. This design expands the number of sequences that can be targeted, by including GA and GAG motifs.

Pyrrole-Imidazole drugs generally bind the DNA minor groove as antiparallel 2:1 drug to DNA complexes (White et al., 1997). However, the affinity cleavage experiments presented here suggest a 1:1 drug to DNA complex both for oligopyrrole dimer Lex18E and P31E (FIGS. 3B and C). In case of Lex18E, this binding mode may be favored by inherent, structural features of long AT-tracts; such runs are known to have a narrower than normal DNA minor groove (Coll et al., 1987). Since binding of two antiparallel oriented molecules requires the expansion of the minor groove (Kielkopf et al., 1998)., widening the AT-tract might energetically be too costly. Likewise, crystal structures of B-DNA oligomers demonstrated that GpA steps tend to narrow the minor groove more than GpT steps (Yanagi et al., 1991) which in turn may disfavor 2:1 complexes between P31 and GAGAA repeats.

Epifuorescent microscopy

Fluorescent DNA dyes with sequence preference, such as DAPI or Hoechst, are useful, everyday tools of cell biology, medicine and cytogenetics. Sequence specific compounds, if successfully rendered fluorescent, could extend the scientific potential enormously, since innumerable basic questions about chromosome structure, function and dynamics could be addressed using sequence specific dyes. Also, such molecules could facilitate and improve more routine work such as chromosome typing.

Although conjugation of a fluorescent label either at the N- or C-terminal end of oligopyrroles is straightforward, tagging at these positions altered affinity (Table 3). In general, tagging reduced binding affinity more on W9 than on SAR thereby improving the SAR specificity factor. For Lex9, this value increased from 2 to 25 and increased from 1.4 to 3 for P9 (Table 3). Both dyes highlight conspicuous foci in Kc nuclei that are proposed to arise from staining of the AT-rich Drosophila satellites I and III. Satellite I, an AATAT repeat, was positively identified by staining of spread polytene chromosomes since the localization of the two major Lex9F signals (at the base of chromosome 4 and 3R) coincided with the known location of satellite I.

The intensity of the staining signal of the foci in nuclei is similar for either dye, in contrast to that of the nucleoplasm. The latter signal was found to be considerably stronger with P9F than Lex9F, which is visually manifested by the greener appearance of the nucleoplasm stained with P9F (FIGS. 4A-C). Quantitatively, on 256 gray scale levels, the average pixel intensity of the nucleoplasm of the green channel is about 130 for P9F and 30 for Lex9F. This visual difference is proposed to reflect qualitatively the binding properties of P9F and Lex9F. Statistically, a W9 tract is 64 times more frequent (every 512 bp) than a W15 run (every 32768 bp). Thus, since P9F, but not Lex9F, binds short and long AT-tracts similarly, a stronger nucleoplasmic signal is expected for P9F.

The reduced nucleoplasmic signal of Lex9F may not only arise from a lower abundance of long/clustered AT-tracts but also from a subnuclear positioning (compartmentalization) of SARs. We previously discovered in mitotic chromosomes an AT-rich subregion, called AT-queue and proposed that it arose from tethering of SARs by the scaffolding (Saitoh and Laemmli, 1994). The subnuclear organization of SARs in nuclei is unknown, but these compounds might well be suited to shed light on this question. Indeed, preliminary visual inspection of nuclei stained with Lex9F or Lex10F is consistent with a non-random SAR organization (not shown). Since three-dimensional reconstruction of differentially stained nuclei demands a much more detailed analysis which will be dealt with in a separate study.

SARs can easily been observed as striking, yellow/green stripes along the euchromatic arms of polytene chromosomes. It will be of interest to correlate this SAR pattern to the Drosophila genome sequence. For this purpose, sequence landmarks are required to position SAR-stripes precisely since currently available cytological maps are not sufficiently precise for this analysis.

The main nuclear targets of P31 were also demonstrated by staining isolated Kc nuclei and polytene chromosomes with the Texas red derivative, P31T. This conspicuously highlighted foci in Kc nuclei that did not overlap with Lex9F signals. These P31T foci must represent the GAGAA repeats of the centric satellite V (FIGS. 6A-C). Positive identification of the main DNA target of P31T was obtained by staining of bwD polytene chromosome whose GAGAA repeat was sharply highlighted by this compound (FIG. 6D). No other P31 signals were observed along the euchromatic arms or at the chromocenter of polytene chromosomes derived from bwD or Canton S. flies. The repetitiveness of these satellite sequences and the polyteny of these chromosomes facilitate the detection of the staining signals. Labeling chromosomes with sequence-specific polyamides is experimentally straightforward, allowing the application of such dyes in innumerable scientific and diagnostic applications. Polytene chromosomes represent an ideal object to asses the specificity of sequence-specific hairpin polyamides.

Chromosome condensation

As in the case of MATH20, Lex10 (but not P9) inhibited chromosome condensation in Xenopus egg extracts specifically. The specificity argument is based on de-repression experiments with different oligonucleotides. Lex10 inhibition could be overcome by addition of a SAR-like oligonucleotide but not by oligonucleotides containing either a W9 tract or AAGAG repeats. The failure to overcome P9 inhibition with either oligonucleotide may be related to the high abundance of short AT-tracts throughout the genome. As mentioned, W9 tracts are statistically 64 times more frequent than W15 tracts. Consequently, a much higher amount of competitor oligonucleotide would be needed to displace P9 from the genome, but are higher concentrations oligonucleotide were found to interfere with chromosome condensation.

Inhibition of chromosome condensation required a Lex10 concentration of about 250 nM, or 80 fold higher than that of MATH20 (3 nM, (Strick and Laemmli, 1995). This is not unexpected, since the affinity of Lex10 for SAR is also approximately 100 lower than that of MATH20. The competition experiment strongly suggests that inhibition of condensation by Lex10 is specific and mediated by SARs. These observations confirm our previous conclusions, implicating SARs in mitotic chromosome structure but do not further extend these data. In addition, these results demonstrate that is possible to synthesize MATH-like compounds of low molecular weight (2.4 kDa vs. 92 kDa).

Chromatin opening

The chromatin studies revealed that titration of AT-tracts with oligopyrrole P9 massively unfolds the heterochromatic satellite Ill. Chromatin opening of satellite III is evidenced by the massive stimulation of cleavage by endogenous topoisomerase II when Kc nuclei were exposed to Xenopus egg extracts. Similar, although less pronounced observationsn have previously been made using distamycin. Unfolding might therefore arise from a displacement of histone H1 or another protein from the nucleosomal linker region (Kas and Laemmli, 1992; Kas et al., 1993). Alternatively, minor groove contacts of the core histones could be of importance for maintaining the heterochromatic state of the chromatin fiber. In contrast to P9, chromatin opening of satellite III required high concentrations of compound P31. In contrast to this, P31 but not P9 can open the heterochromatic GAGAA insert which constitutes the brown-dominant allele (be) (data not shown). These observations suggest the DNA minor groove binding polyamides may serve as sequence-specific chromatin openers for silenced genes.

Lex10 did not open chromatin, but in contrast, it efficiently blocked cleavage by topoisomerase II in a satellite-specific fashion since the genome-wide fragmentation mediated by this activity was not inhibited. Previous studies showed that netropsin dimers were also more potent, general (not sequence-specific) inhibitors of this enzyme than monomers (Beerman et al., 1991). Topoisomerase II cleavage occurs in satellite III in a 10 bp GC-rich batch that is flanked by very AT-rich (85 to 90%) DNA (Kas and Laemmli, 1992). Lex10 could possibly sterically block cleavage by positioning its hooks in the flanking AT-rich regions and spanning the central GC-rich patch with its long linker. Topoisomerase II is a prominent target for anticancer drugs, perhaps a sequence-specific such as Lex10, rather than general inhibitor of this activity, may have interesting potentials in this respect.

These experiments identify sequence-specific polyamides as very powerful tools for chromosome research.

### REFERENCES

Adachi, Y., Kas, E., and Laemmli, U. K. (1989). Preferential, cooperative binding of DNA topoisomerase II to scaffold-associated regions. Embo J 8, 3997-4006.

Adachi, Y., Luke, M., and Laemmli, U. K. (1991). Chromosome assembly in vitro: topoisomerase II is required for condensation. Cell 64, 137-48.

Baird, E. E., and Dervan, P. B. (1996). Solid phase synthesis of polyamides containing imidazole and pyrrole amino acids. J Am Chem Soc 118, 6141-6146.

Beerman, T. A., Woynarowski, J. M., Sigmund, R. D., Gawron, L. S., Rao, K. E., and Lown, J. W. (1991). Netropsin and bis-netropsin analogs as inhibitors of the catalytic activity of mammalian DNA topoisomerase II and topoisomerase cleavable complexes. Biochim Biophys Acta 1090, 52-60.

Biggin, M. D., Bickel, S., Benson, M., Pirrotta, V., and Tjian, R. (1988). Zeste encodes a sequence-specific transcription factor that activates the Ultrabithorax promoter in vitro. Cell 53, 713-22.

Bode, J., Kohwi, Y., Dickinson, L., Joh, T., Klehr, D., Mielke, C., and Kohwi-Shigematsu, T. (1992). Biological significance of unwinding capability of nuclear matrix-associating DNAs. Science 255, 195-7.

Boy de la Tour, E., and Laemmli, U. K. (1988). The metaphase scaffold is helically folded: sister chromatids have predominantly opposite helical handedness. Cell 55, 937-44.

Coll, M., Frederick, C. A., Wang, A. H., and Rich, A. (1987). A bifurcated hydrogen-bonded conformation in the d(A.T) base pairs of the DNA dodecamer d(CGCAAATTTGCG) and its complex with distamycin. Proc Natl Acad Sci USA 84, 8385-9.

Csink, A. K., and Henikoff, S. (1996). Genetic modification of heterochromatic association and nuclear organization in Drosophila. Nature 381, 529-31.

de Clairac, R. P. L., Seel, C. J., Geierstanger, B. H., Mrksich, M., Baird, E. E., Dervan, P. B., and Wemmer, D. E. (1999). NMR characterization of the aliphatic b/b pairing for recognition of AT/TA base pairs in the minor groove of DNA. J Am Chem Soc 121, 2956-2964.

Dernburg, A. F., Broman, K. W., Fung, J. C., Marshall, W. P., Philips, J., Agard, D. A., and Sedat, J. W. (1996). Perturbation of nuclear architecture by long-distance chromosome interactions. Cell 85, 745-59.

Dimitrov S, D. M., Wolffe A P (1994). Remodeling sperm chromatin in Xenopus laevis egg extracts; the role of core histone phosphorylation and linker histone B4 in chromatin assembly. J Cell Biol 126, 591-601.

Forrester, W. C., Fernandez, L. A., and Grosachedl, R. (1999). Nuclear matrix attachment regions antagonize methylation-dependent repression of long-range enhancer-promoter interactions. Genes Dev 13, 3003-3014.

Frederickson, R. (1999). “Functional” proteomics? Nat Biotechnol 17, 1050.

Gasser, S. M., and Laemmli, U. K. (1986). Cohabitation of scaffold binding regions with upstream/enhancer elements of three developmentally regulated genes of D. melanogaster. Cell 46, 521-30.

Geierstanger, B. H., Mrksich, M., Dervan, P. B., and Wemmer, D. E. (1994). Design of a G.C-specific DNA minor groove-binding peptide. Science 266, 646-50.

Girard, P., Bello, B., Laemmli, U. K., and Gehring, W. J. (1998). In vivo analysis of scaffold-associated regions in Drosophila; a synthetic high-affinity SAR binding protein suppresses position effect variegation. Embo J 17, 2079-85.

Goodsell, D., and Dickerson, R. E. (1986). Isohelical analysis of DNA groove-binding drugs. J Med Chem 29, 727-33.

Gottesfeld, J. M., Neely, L., Trauger, J. W., Baird, E. E., and Dervan, P. B. (1997). Regulation of gene expression by small molecules. Nature 387, 202-5.

Hart, C. M., and Laemmli, U. K. (1998). Facilitation of chromatin dynamics by SARs. Curr Opin Genet Dev 8, 519-25.

Henikoff, S. (2000). Heterochromatin function in complex genomes. Biochim Biophys Acta 1470, 01-08.

Hirano, T., and Mitchison, T. J. (1991). Cell cycle control of higher-order chromatin assembly around naked DNA in vitro. J Cell Biol 115, 1479-89.

Hirano T. K. R., Hirano M (1997). Condensins, chromosome condensation protein complexes containing XCAP-C, XCAP-E and a Xenopus homolog of the Drosophila Barren protein. Cell 89, 511-21.

Hsieh, T., and Brutlag, D. (1979). Sequence and sequence variation within the 1.688 g/cm3 satellite DNA of Drosophila melanogaster. J Mol Biol 135, 465-81.

Janssen, S., Cuvier, O., Muller, M., and Laemmli, U. K. (2000). Specific Gain and Loss of Function Phenotypes induced by Satellite-specific DNA-binding Drugs fed to Drosophila melanogaster.

Karpen, G. H. (1994). Position-effect variegation and the new biology of heterochromatin. Curr Opin Genet Dev 4, 281-91.

Kas, E., and Laemmli, U. K. (1992). In vivo topoisomerase II cleavage of the Drosophila histone and satellite III repeats: DNA sequence and structural characteristics. Embo J 11, 705-16.

Kas, E., Poljak, L., Adachi, Y., and Laemmli, U. K. (1993). A model for chromatin opening; stimulation of topoisomerase II and restriction enzyme cleavage of chromatin by distamycin. Embo J 12, 115-26.

Kielkopf, C. L., Baird, E. B., Dervan, P. B., and Rees, D. C. (1998). Structural basis for G.C recognition in the DNA minor groove. Nat Struct Biol 5, 104-9.

Kirillov, A., Kistler, B., Mostoslavsky, R., Cedar, H., Wirth, T., and Bergman, Y. (1996). A role for nuclear NF-kappaB in B-cell-specific demethylation of the Igkappa locus. Nat Genet 13, 435-41.

Laemmli, U. K., Kas, E., Poljak, L., and Adachi, Y. (1992). Scaffold-associated regions: cis-acting determinants of chromatin structural loops and functional domains. Curr Opin Genet DeV 2, 275-85.

Lohe, A. R., Hilliker, A. J., and Roberts, P. A. (1993). Mapping simple repeated DNA sequences in heterochromatin of Drosophila melanogaster. Genetics 134, 1149-74.

Marshall, W. F., Straight, A., Marko,, J. F., Swedlow, J., Dernburg, A., Belmont, A., Murray, A. W., Agard, D. A., and Sedat, J. W. (1997). Interphase chromosomes undergo constrained diffusional motion in living cells. Curr Biol 7, 930-9.

Martello, P. A., Bruzik, J. P., deHaseth, P., Youngquist, R. S., and Dervan, P. B. (1989). Specific activation of open complex formation at an Escherichia coli promoter by oligo(N-methylpyrrolecarboxamide)s: effects of peptide length and identification of DNA target sites. Biochemistry 28, 4455-61.

McBryant, S. J., Baird, E. E., Trauger, J. W., Dervan, P. B., and Gottesfeld, J. M. (1999). Minor groove DNA-protein contacts upstream of a tRNA gene detected with a synthetic DNA binding ligand. J Mol Biol 286, 973-81.

Miklos, G. L., and Cotsell, J. N. (1990): Chromosome structure at interfaces between major chromatin types: alpha- and beta-heterochromatin. Bioessays 12, 1-6.

Mirkovitch, J., Mirault, M. E., and Laemmli, U. K. (1984). Organization of the higher-order chromatin loop: specific DNA attachment sites on nuclear scaffold. Cell 39, 223-32.

Neamati, N., Mazumder, A., Sunder, S., M., O. J., M., T., Lown, J. W., and Pommier, Y. (1998). Highly potent synthetic polyamides, bisdistamycins, and lexitropsins an inhibitors of human immunodeficiency virus type 1 integrase. Mol Pharmacol 54, 280-90.

Omichinski, J. G., Pedone, P. V., Felsenfeld, G., Gronenborn, A. M., and Clore, G. M. (1997). The solution structure of a specific GAGA factor-DNA complex reveals a modular binding mode [see comments]. Nat Struct Biol 4, 122-32.

Pelton, J. G., and Wemmer, D. E. (1989), Structural characterization of a 2:1 distamycin A.d(CGCAAATTGGC) complex by two-dimensional NMR. Proc Natl Acad Sci U S A 86, 5723-7.

Platero, J. S., Csink, A. K., Quintanilla, A., and Henikoff, S. (1998). Changes in chromosomal localization of heterochromatin-binding proteins during the cell cycle in Drosophila. J Cell Biol 140, 1297-306.

Reeves, R., and Nissen, M. S. (1990). The A.T-DNA-binding domain of mammalian high mobility group I chromosomal proteins. A novel peptide motif for recognizing DNA structure. J Biol Chem 265, 8573-92.

Rykowski, M. C., Parmelee, S. J., Agard, D. A., and Sedat, J. W. (1988). Precise determination of the molecular limits of a polytene chromosome band: regulatory sequences for the Notch gene are in the interband. Cell 54, 461-72.

Saitoh, Y., and Laemmli, U. K. (1994). Metaphase chromosome structure: bands arise from a differential folding path of the highly AT-rich scaffold. Cell 76, 609-22.

Spierer, A., and Spierer, P. (1984). Similar level of polyteny in bands and interbands of Drosophila giant chromosomes. Nature 307, 176-8.

Strick, R., and Laemmli, U. K. (1995). SARs are cis DNA elements of chromosome dynamics: synthesis of a SAR repressor protein. Cell 83, 1137-48.

Taylor, J. S., Schultz, P. G., and Dervan, P. B. (1984). Sequence specific cleavage of DNA by distamycin-EDTA Fe(II) and EDTA-distamycin Fe(II). Tetrahedron 40, 457-465.

Turner, J. M., Baird, E. E., and Dervan, P. P. (1997). Recognition of seven base pair sequences in the minor groove of DNA by ten-ring pyrrole-imidazole polyamide hairpins. J. Am. Chem. Soc. 119, 7636-7644.

Turner, J. M., Swalley, S. E., Baird, E. E., and Dervan, P. B. (1998). Aliphatic/aromatic amino acid pairings for polyamide recognition in the minor groove of DNA. J. Am. Chem. Soc. 120, 6219-6226.

Wang, W., and Lown, J. W. (1992). Anti-HIV-I activity of linked lexitropsins. J Med Chem 35, 2890-7.

White, S., Baird, E. E., and Dervan, P. B. (1997). On the pairing rules for recognition in the minor groove of DNA by pyrrole-imidazole polyamides. Chem Biol 4, 569-78.

Yanagi, K., Privé, G. G., and Dickerson, R. E. (1991). Analysis of local helix geometry in three B-DNA decamers and eight dodecamers. J. Mol. Biol. 217, 201-214.

Youngquist, R. S., and Dervan, P. B. (1985). Sequence-specific recognition of B-DNA by oligo(N-methylpyrrolecarboxamide)s. Proc Natl Acad Sci USA 82, 2565-9.

Youngquist, R. S., and Dervan, P. B. (1987). A synthetic Peptide binds 16 base pairs of A,T double helical DNA. J Am Chem Soc 109, 564-7566.

TABLE 3
Apparent Binding Affinities of Oligopyrroles

| | | Kdₐₚₚ | Kdₐₚₚ | |
|---|---|---|---|---|
| | | W9 | SAR | |
| Compound | Sequence | (nM) | (nM) | Ratio |
| P10 | (Py)₅-β-Dp | 80 | 35 | 2.3 |
| P9 | (Py)₃β-(Py)₃-β-Dp | 0.75 | 0.55 | 1.4 |
| P13 | ((Py)₃-β)₃-Dp | 1.0 | 1.25 | 0.8 |
| Lex9 | (Py)₅-β-Dp-Glu-(Ao)₃-(Py)₅- | 3.5 | 1.75 | 2 |
| | β-Dp | | | |
| Lex10 | (Py)₃-β-(Py)₃-β-Dp-Glu- | 20 | 0.28 | 71 |
| | (Ao)₃(Py)₅-β-Dp | | | |
| Lex18 | (Py)₃-β-(Py)₃-(Ao)₃-(Py)₅-β-Dp | 100 | 1.0 | 100 |
| P9F | (Py)₃-β-(Py)₃-β-Dp-F* | 4.0 | 1.0 | 4 |
| P10F | F*-γ-(Py)₅-β-Dp | 3000 | 1200 | 2.5 |
| Lex9F | F*-γ-(Py)₅-β-Dp-Glu-(Ao)₃- | 2500 | 100 | 25 |
| | (Py)₅-β-Dp | | | |
| Lex10F | F*-γ-(Py)₃-β-(Py)₃-β-Dp-Glu- | 20 | 1.0 | 20 |
| | (Ao)₃-(Py)₅-β-Dp | | | |

Ao = 8 amino 3,6 dioxactanoic acid
γ = 4 amino butyric acid
β = beta alanine
F* = Fluorescein

1. DNA-binding molecule, capable of sequence specific binding to the minor groove of double-stranded DNA, characterised in that it comprises at least two sequence specific DNA-binding elements, covalently linked to each other in tandem orientation by an amphipathic, flexible linker molecule, at least one of said DNA binding elements being non-proteinaceous.

2. DNA-binding molecule according to claim 1 wherein at least one of the DNA-binding elements comprises an oligomer comprising one or more organic heterocyclic amino-acid residues.

3. DNA-binding molecule according to claim 2 wherein each organic heterocyclic residue has at least one annular nitrogen, sulphur or oxygen.

4. DNA-binding molecule according to claim 2 or 3 wherein said heterocyclic residue is chosen from pyrrole, imidazole, triazole, pyrazole, furan, thiazole, thiophene, oxazole, pyridine, or derivatives of any of these compounds wherein one or more of the heteroatoms are substituted by a substituent which is DNA-binding or non-DNA-binding.

5. DNA-binding molecule according to claim 4 wherein at least one oligomer includes heterocyclic residues chosen from N-methylpyrrole (Py) and/or 3-hydroxy N-methylpyrrole (Hp) and/or N-methylimidazole Im).

6. DNA-binding molecule according to any one of claims 2 to 5 wherein the DNA-binding element further comprises at least one aliphatic amino acid residue such as a β-alanine (β) residue, or a 5-aminovaleric acid residue.

7. DNA-binding molecule according to any one of claims 1 to 4, having the general formula (I): 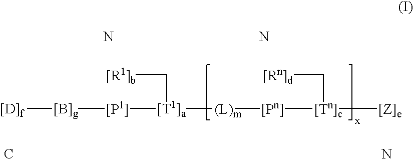
wherein
each of P¹ to Pⁿ represents a DNA-binding element, said element comprising multiple organic heterocyclic or aliphatic residues or fluorescent derivatives thereof;
each of R¹ to Rⁿ represents a DNA-binding element, said element comprising multiple organic heterocyclic or aliphatic residues or fluorescent derivatives thereof;
x represents an integer from 1 to 20, with the proviso that when x is greater than 1, the multiple copies of [Rⁿ], [Lⁿ], [Pⁿ] and [Tⁿ] may be the same or different;
n represents an integer having a value equal to (x+1);
[T] represents a multifunctional linking molecule providing a covalent link between DNA-binding elements [R] and [P], with the proviso that if “e” represents 0, [T^{x+1}] can be bifunctional;
each of a and c independently represent 0 or 1;
each of b and d independently represent 0 or 1, with the proviso that when a represents 0, b also represents 0, and when c represents 0, d also represents 0;
[D] represents an end group or an effector moiety,
[L]ₘ represents an amphipathic, flexible linker molecule, linking the DNA-binding elements in a tandem orientation with respect to each other;
m represents an integer from 1 to 10;
[B] represents a spacer unit such as β-alanine;
[Z] represents an end group or an effector moiety;
each of f, g and e independently represent 0 or 1,
each solid line represents a covalent bond;
N and C indicate the N- and C-terminal extremities of the molecules respectively.

8. DNA-binding molecule according to claim 7 wherein the DNA-binding elements P and R comprise heterocyclic residues chosen from pyrrole, imidazole, triazole, pyrazole, furan, thiazole, thiophene, oxazole, pyridine, or derivatives of any of these compounds wherein one or more of the heteroatoms is substituted.

9. DNA-binding molecule according to claim 8 having the general formula (II): 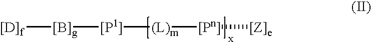
wherein [P¹], [Pⁿ], (L), [D], [Z], x, m, f, g and e have the previously defined meanings
and a dotted line represents a covalent bond which can be present or absent.

10. DNA-binding molecule according to claim 9 wherein each of the the DNA-binding elements [P¹] to [Pⁿ] independently have the general formula (III)
—[U²-[U]ₛ]— (III)
wherein
each U is independently a monomeric unit chosen from a heterocyclic amino acid residue, or an aliphatic amino acid residue or a fluorescent derivative thereof, and s is an integer from 1 to 15, preferably from 2 to 8,
and a dotted line represents a covalent bond which can be present or absent.

11. DNA-binding molecule according to claim 10 wherein at least one U is chosen from N-methylpyrrole (Py) and/or 3-hydroxy N-methylpyrrole (HP) and/or N-methylimidazole (Im).

12. DNA-binding molecule according to claim 10 wherein at least one U is a β-alanine (β) residue, or a 5-aminovaleric acid residue.

13. DNA-binding molecule according to claim 11 or 12 wherein S is an integer from 2 to 5

14. DNA-binding molecule according to claim 13 wherein at least one of [P¹] to [Pⁿ] comprises between 3 to 5 heterocyclic amino acid residues.

15. DNA-binding molecule according to claim 13 wherein at least one of [P¹] to [Pⁿ] comprises more than two contiguous heterocyclic amino acid residues, for example three, four or five contiguous heterocyclic amino acid residues.

16. DNA-binding molecule according to claim 15 wherein stretches of three to five contiguous heterocyclic amino acid residues are separated from each other by a β-alanine residue

17. DNA-binding molecule according to claim 10 wherein at least one of [P¹] to [Pⁿ] has the formula (IV) 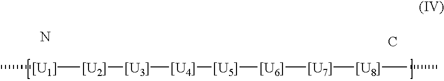
wherein U is as previously defined,
[U₄] is β-alanine,
[U₁] to [U₃], and [U₅] to [U₇] are chosen from N-methylpyrrole
(Py) and/or N-methylimidazole (Im),
[U₈] may be present or absent, and it present is preferentially β-alanine,
and a dotted line represents a covalent bond which can be present or absent.

18. DNA-binding molecule according to claim 17 wherein [U₁] to [U₃], and [U₅] to [U₇] are each N-methylpyrrole (Py).

19. DNA-binding molecule according to claim 10 wherein at least one of [P¹] to [Pⁿ] has the fomula (V): 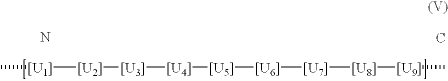
wherein
U is as previously defined,
[U₁] to [U₈] are chosen from N-methylpyrrole (Py), N-methylimidazole (Im) and a β alanine residue,
with the proviso that the [U] immediately adjacent to each Im on the N-terminal side is a β alanine residue, [U₉] may be present or absent, and if present is preferentially β-alanine,
and a dotted line represents a covalent bond which can be present or absent.

20. DNA-binding molecule according to claim 19 wherein at least one of [P¹] to [Pⁿ] has the formula (VI):

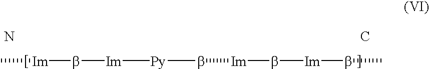

21. DNA-binding molecule according to any one of claims 9 to 20 wherein x represents a value from 2 to 10, for example 2, 3, 4, 5, 6, 7, 8, 9, or 10.

22. DNA-binding molecule according to claim 7 having the general formula (VII): 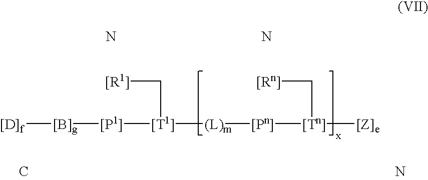
wherein [R¹], [P¹], [Rⁿ], [Pⁿ], [T¹], [Tⁿ], (L) [D], [B], [Z], m, n, g, f and e have the previously defined meanings.

23. DNA-binding molecule according to claim 22 wherein each of the DNA-binding elements [P¹] to [Pⁿ] and [R¹] to [Rⁿ] independently have the general formula (VIII)
—[U¹-[U]ₛ]— (III)
wherein:
each U is independently a monomeric unit chosen from a heterocyclic amino acid residue, or an aliphatic amino acid residue or a fluorescent derivative of the foregoing, and s is an integer from 0 to 15, preferably from 1 to 6
and a dotted line represents a covalent bond which can be present or absent.

24. DNA-binding molecule according to claim 23 wherein at least one heterocyclic amino acid residue comprises an annular nitrogen.

25. DNA-binding molecule according to claim 24 wherein at least one of [P¹] to [Pⁿ] or [R¹] to [Rⁿ] contain a residue of N-methylpyrrole (Py) and/or 3-hydroxy. N-methylpyrrole (HP) and/or N-methylimidazole (Im).

26. DNA-binding molecule according to claim 25 wherein at least one of [P¹] to [Pⁿ] or [R¹] to [Rⁿ] contain an aliphatic amino-acid residue such as a β-alanine (β) residue.

27. DNA-binding molecule according to claim 23 or 24 wherein s is an integer from 2 to 6.

28. DNA-binding molecule according to claim 23 or 24 wherein at least one of [P¹] to [Pⁿ] or [R¹] to [Rⁿ] comprises at least four heterocyclic amino acid residues.

29. DNA-binding molecule according to claim 23 wherein at least one of [P¹] to [Pⁿ] or [R¹] to [Rⁿ] comprises more than two contiguous heterocyclic amino acid residues, for example three, four or five contiguous heterocyclic amino acid residues.

30. DNA-binding molecule according to claim 29 wherein stretches of three to five contiguous heterocyclic amino acid residues are separated from each other by a β-alanine residue.

31. DNA-binding molecule according to claim 23 wherein at least one [Pⁿ] element has the formula (IX): 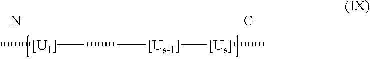
and at least one [Rⁿ] element has the formula (X): 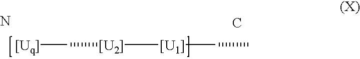
wherein each U represents independently N-methylpyrrole (Py), or 3-hydroxy N-methylpyrrole (HP), or N-methylimidazole (Im) or N-methyl pyrazole (Pz), or 3-pyrazolecarboxylic acid (3-Pz), or β-alanine (β), q and s are independently integers from 1 to 10,
and a dotted line represents a covalent bond which can be present or absent,
wherein the U residues of [Pⁿ] form anti-parallel pairs with the U residues of [Rⁿ]: 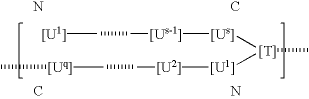
said pairs being chosen from Py/Im, Im/Py, Py/Py, Hp/Py, Py/Hp, β/Py, Py/β, β/Im, Im/β, Im/Im, Pz/Py, 3-Pz/Pz, and β/β.

33. DNA-binding molecule according to claim 9 wherein at least one DNA-binding element contains [T], [R] and [P] moieties, and at least one DNA binding element is free of [T] and [R] moieties.

34. DNA-binding molecule according to claim 9 wherein the multiple [R] and [P] elements are different in length and/or composition.

35. DNA-binding molecule according to any one of the preceding claims 7 to 34 having the capacity to bind in a multidentate mode to a given strand of DNA.

36. DNA-binding molecule according to any one of the preceding claims 7 to 35 wherein m represents a value greater than or equal to one, and the amphipathic linker (L)ₘ comprises an assembly of linker sub-units (L).

37. DNA-binding molecule according according to claim 36 wherein the assembled linker (L)ₘ is heterobifunctional.

38. DNA-binding molecule according to claim 36 wherein each linker sub-unit (L) is heterobifunctional.

39. DNA-binding molecule according to claim 36, wherein at least one (L) sub-unit is amphipathic.

40. DNA-binding molecule according to claim 37 wherein the total length of the linker (L)ₘ is between 5 to 250 Angstroms, for example 5 to 50 Angstroms.

41. DNA-binding molecule according to claim 37 wherein the functional groups are chosen from amino, carboxyl, thiol, haloacetyl, aldehyde, amino-oxy, maleimide groups, a symetrical anhydride and halogen atoms.

42. DNA-binding molecule according to claim 36 wherein at least one amphipathic linker (L) sub-unit comprises one or more ether groups and/or ester groups for example molecules derived from ethylene oxide or propylene oxide.

43. DNA-binding molecule according to claim 36 wherein at least one amphipathic linker (L) sub-unit comprises one or more units of 8-amino-3,6-dioxaoctanoic acid (Ao).

44. DNA-binding molecule according to any one of claims 1 to 43 having the capacity to bind in a sequence specific manner to a DNA recognition sequence of at least 6, preferably at least 10 and most preferably at least 14 base pairs in length.

45. DNA-binding molecule according to any one of claims 1 to 44 having a molecular weight no greater than approximately 8 kDa.

46. DNA-binding molecule according to any one of claims 1 to 45 wherein the said molecule binds to the DNA minor groove.

47. DNA-binding molecule according to any one of claims 1 to 46 which is cell-permeable.

48. DNA-binding molecule according to any one of claims 1 to 47 having an apparent binding affinity of at least 5×10⁷M⁻¹.

49. DNA-binding molecule according to any one of claims 1 to 48 having an apparent binding affinity of at least 1×10⁹M⁻¹.

50. DNA-binding molecule according to any one of claims to 49 having an apparent binding affinity of at least 5×10¹⁰M⁻¹.

51. Process for binding double-stranded DNA in a sequence-specific manner, comprising contacting a DNA-target sequence within said DNA with a DNA-binding molecule according to any one of claims 1 to 50, in conditions allowing said binding to occur.

52. Process according to claim 51 which is carried out in vivo, in vitro or ex vivo.

53. Process according to claim 52 which is carried out in a cell.

54. Process according to claim 53, wherein said cell is eukaryotic.

55. Process according to claim 53, wherein said cell is prokaryotic.

56. Process according to claim 54, wherein said cell is a vertebrate cell, an invertebrate cell, a plant cell

57. Process according to claim 54, wherein said cell is a mammalian cell, an insect cell, or a yeast cell.

58. Process according to any one of claims 51 to 57 wherein the double stranded DNA is endogenous to said cell.

59. Process according to any one of claims 51 to 57 wherein the double stranded DNA is heterologous to said cell.

60. Process according to claim 53 wherein the double stranded DNA target sequence comprises a chromatin element.

61. Process according to claim 60 wherein the target sequence comprises a SAR-like sequence.

62. Process according to claim 60 wherein the target sequence comprises a GAGAA repeat sequence.

63. Process according to any one of claims 50 to 62 wherein the target sequence has at least 8 and preferably at least 15 bases.

64. Process according to claim 60 wherein the target sequence is a cis- or trans-acting element mediating chromosome function.

65. Process according to claim 64 wherein the binding of the target element with the sequence-specific binding molecule gives rise to cis- and/or trans-regulation of chromosome function.

66. Process according to claim 53 wherein the double stranded DNA target sequence comprises a site mediating the activity of one or more regulatory factors.

67. Process according to claim 66 wherein the regulatory factors is a transcription regulatory factor, a DNA replication factor, a factor for enzymatic activity, a factor involved in chromosome stability.

68. Process according to-any one of claims 51 to 67, wherein the DNA-binding molecule is linked to an effector moiety.

69. Process for modulating chromosome function in a eukaryotic cell,
comprising the step of contacting a genomic DNA element,
comprising a binding site mediating chromosome function,
with a molecule according to any one of claims 1 to 50 and having the capacity to bind in a sequence-specific manner to said element,
said step of contacting being carried out in conditions permitting binding of said compound to-said element,
wherein the binding modulates chromosome function.

70. Process for modulating the function of a DNA element in a eukaryotic cell,
Comprising the step of contacting a genomic DNA element, so-called <<chromatin responsive element>>0 (CRE),
with a molecule according to any one of claims 1 to 50 and having the capacity to bind in a sequence-specific manner to said CRE,
said step of contacting being carried out in conditions permitting chromatin remodeling of the CRE by said compound,
wherein said chromatin remodeling of the CRE alters the activity of one or more other DNA elements, so called <<modulated DNA elements>> in the genome.

71. Cell containing a compound according to any one of claims 1 to 50.

72. Cell according to claim 71, wherein said compound binds the DNA-minor groove.

73. Cell according to claim 71 which is a eukaryotic cell.

74. Non-human organism comprising a cell according to claim 71.

75. Organism according to claim 74 which is a non-human animal.

76. Organism according to claim 75 which is a transgenic, non-human animal.

77. Organism according to claim 74 which is a plant.

78. Organism according to claim 77 which is a transgenic plant.

79. Pharmaceutical composition comprising a compound according to any one of claims 1 to 50 in association with a physiologically acceptable excipient.

80. Compound according to any one of claims 1 to 50, for use in therapy.

81. Compound according to any one of claims 1 to 50 which is fluorescent or fluorescently labelled.

82. Compound according to claim 81 wherein the fluorescent label is a fluorescent dye such as fluorescein, dansyl, Texas red, isosulfan blue, ethyl red, malachite green, rhodamine and cyanine dyes.

83. Use of a compound according to claim 81 for probing the epigenetic state and location of DNA in chromosomes and nuclei.

84. Use according to claim 83 for diagnosis of pathological conditions arising from epigenetic status.

85. Use of a compound according to claim 81 for chromosome visualisation and marking in diagnosis, forensic studies, affiliation studies, or animal husbandry.
